(19) United States
(12) Patent Application Publication
(10) Pub. No.: US 2017/0164829 A1
(43) Pub. Date: Jun. 15, 2017

(54) Registration Using a Microscope Insert

(71) Applicant: Richard AWDEH, Miami, FL (US)

(72) Inventor: Richard AWDEH, Miami, FL (US)

(73) Assignee: Nanophthalmos, LLC, Miami, FL (US)

(21) Appl. No.: 15/123,036

(22) PCT Filed: Mar. 13, 2015
(86) PCT No.: PCT/US15/20595

§ 371 Date: Sep. 1, 2016

Related U.S. Application Data
(60) Provisional application No. 61/952,816, filed on Mar. 13, 2014.

(51) Int. Cl. A61B 3/13; G02B 21/24; G02B 21/22; A61B 3/14; A61B 34/20; A61B 3/113; G02B 21/00; A61B 90/20; A61B 90/00; G02B 21/36; A61B 3/00

(52) U.S. Cl. CPC A61B 3/132; G02B 21/365; G02B 21/368; G02B 21/244; G02B 21/22; A61B 3/14; A61B 3/0025; A61B 3/0058; A61B 3/113; G02B 21/0012; A61B 90/20; A61B 90/37; A61B 34/20; A61B 2090/3612; A61B 2090/363; A61B 2090/364; A61B 2034/2065

(57) ABSTRACT

A microscope insert includes a camera, a display device, a beam splitter, and a processing unit. The camera is configured to receive a first portion of first light through a microscope from an object and generate a signal representing an image of the object. The display device is configured to generate a graphical representation of information relevant to the object and project second light representing the graphical representation. The beam splitter is configured to direct a second portion of the first light from the object and a first portion of the second light to a viewing device for simultaneously viewing the object and the information by a user. The processing unit is configured to track motions of the object based on the image of the object and control the display device to adjust the graphical representation according to the motions of the object.

### CROSS REFERENCE TO RELATED APPLICATIONS

This application claims the benefit of priority to U.S. Provisional Application No. 61/952,816, filed Mar. 13, 2014.

### TECHNICAL FIELD

This disclosure is related in general to surgical microscopes and in particular to registration using a microscope insert for surgical microscopes.

### BACKGROUND

Surgery carried out through a microscope, such as the cataract surgery, presents special challenges for the surgeon and the microscope. Not only must each procedure and step be carried out accurately, but parameters of the surgery and biological data of the patient must be monitored closely to achieve desired results and ensure safety of the patient. Existing surgical systems, such as ophthalmology microscopes, do not have the ability to display the surgical site and related data within the same field of view. As a result, the surgeon must move away from the eye pieces of the microscope to an external display device in order to view the related data and then move back to the eye pieces in order to continue the surgery. This is not only inconvenient, but may also cause patient safety issues. In addition, existing surgical systems do not provide sufficient prompts or guidance to the surgeon to ensure a correct procedure is carried out. It is desired to provide system-generated prompts for the surgeon during the surgery.

### SUMMARY

According to an embodiment, a microscope insert includes a camera, a display device, a beam splitter, and a processing unit. The camera is configured to receive a first portion of first light through a microscope from an object and generate a signal representing an image of the object. The display device is configured to generate a graphical representation of information relevant to the object and project second light representing the graphical representation. The beam splitter is configured to direct a second portion of the first light from the object and a first portion of the second light to a viewing device for simultaneously viewing the object and the information by a user. The processing unit is configured to track motions of the object based on the image of the object and control the display device to adjust the graphical representation according to the motions of the object.

According to another embodiment, a method for tracking and registering an object in a microscope is disclosed. The method includes receiving first light from an object through a microscope; generating, based on a first portion of the first light, a first signal representing an image of the object; generating, according to the image of the object, a graphical representation of information relevant to the object; projecting second light corresponding to the graphical representation of the information; directing a second portion of the first light from the object and a first portion of the second light to a viewing device for simultaneously viewing the object and the information by a user; tracking the object based on the image of the object; and adjusting the graphical representation according to the tracking of the object.

### BRIEF DESCRIPTION OF THE DRAWINGS

FIG. 1 is a schematic diagram of a microscope insert according to an embodiment;

FIG. 2 illustrates a surgical system including the microscope insert according to an embodiment;

FIG. 3 illustrates electronic connections between the microscope insert and an external computer system according to an embodiment;

FIG. 4 illustrates a microscope system including an microscope insert according to an embodiment;

FIG. 5 illustrates light paths within a microscope insert according to an embodiment:

FIG. 6A is a side view of various components assembled in a microscope insert according to an embodiment;

FIG. 68 is a top view of the various components assembled in the microscope insert according to an embodiment;

FIGS. 7A and 7B are perspective views of a microscope insert having various components installed therein according to an embodiment;

FIG. 8 is a schematic diagram of a microscope insert according to an embodiment;

FIG. 9 is a schematic diagram of an insert driver circuit board for a microscope insert according to an embodiment;

FIG. 10 illustrates graphical information generated by an microscope insert according to an embodiment;

FIG. 11 illustrate a process for correcting a field of view of the microscope insert according to an embodiment; and

FIG. 12 illustrates a process for generating an overlaid image in a microscope according to an embodiment;

FIG. 13 illustrates a process for marker-based registration and tracking according to an embodiment;

FIG. 14 illustrates a process for anatomical feature-based registration and tracking according to an embodiment;

FIG. 15A is an image of an eye with fiducial markers captured by a camera according to an embodiment;

FIG. 15B is an enhanced image of an eye with fiducial markers generated based on the image of FIG. 15A;

FIG. 150 is a binary mask generated based on the enhanced image of FIG. 15B;

FIGS. 16A-16C illustrate a K-means clustering process for segmenting the image captured by a camera of the disclosed microscope insert according to an embodiment;

FIG. 17 is a mask image generated by a K-means clustered image according to the process of FIGS. 16A-16C;

FIG. 18A is a reference image used for feature identification according to an embodiment;

FIG. 16B is a test image captured by a camera for feature identification according to an embodiment;

FIG. 19 illustrates the mapping between the reference image of FIG. 18A and the test image of FIG. 18B according to an embodiment;

FIG. 20 illustrates a sequence diagram for interactions between a tracker engine configured to track the motions of an eye and a torsion engine configured to generate graphical representations for the guidance or prompts;

FIG. 22 illustrates an exemplary process for carrying out the tracking by the tracker engine of FIG. 21;

FIG. 23 illustrates an exemplary process for reference image processing and generating Housdorff Distance look-up table:

FIG. 24 illustrates an exemplary process for processing the sense image and computation of the minimum-Hausdorff Distance between the reference image template ROI and ROI in the sense image; and

FIGS. 25-32 depict a process for estimation of ocular torsion from sclera features.

### DESCRIPTION OF THE EMBODIMENTS

Reference will now be made in detail to exemplary embodiments of the disclosure, examples of which are illustrated in the accompanying drawings. Wherever possible, the same reference numbers will be used throughout the drawings to refer to the same or like parts.

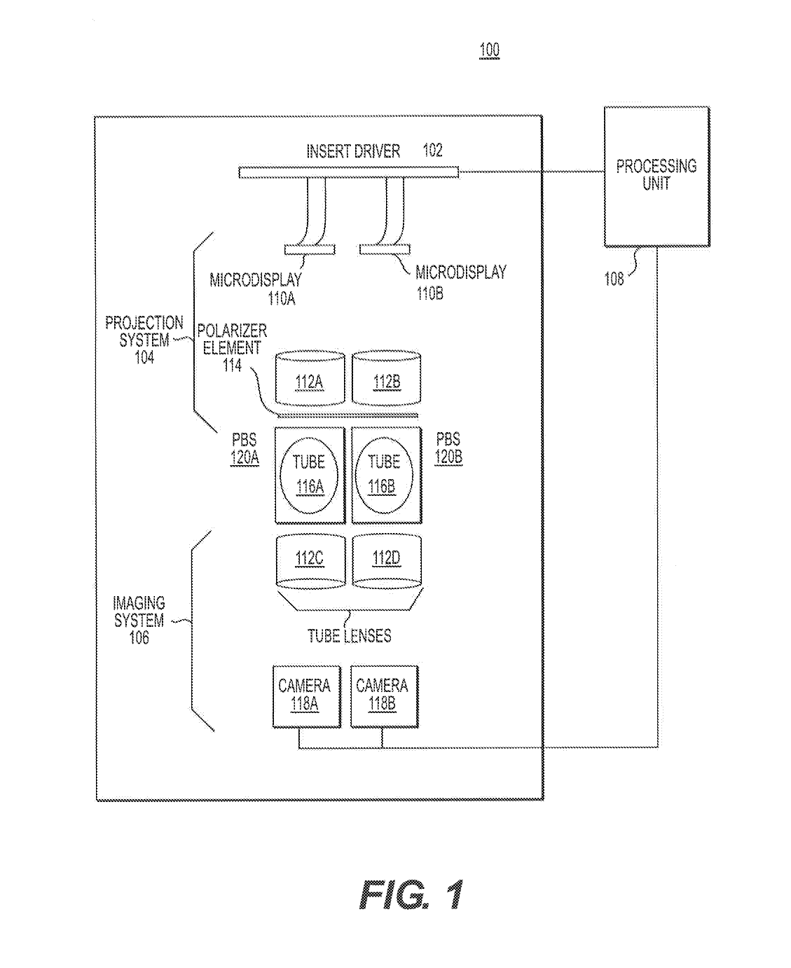

As shown in FIG. 1, a microscope insert 100 includes a projection system 104 and an imaging system 106. Projection system 104 includes one or more display devices 110A and 1108 and one or more sets of tube lenses 112A and 112B for projecting images from the display devices 110A and 110B. Imaging system 106 includes one or more cameras 118A and 118B and one or more sets of tube lenses 112C and 112D for focusing images to cameras 118A and 118B. Microscope insert 100 further includes one or more polarizing beam splitters (PBS) 120A and 120B, which will be further described below.

The above components of insert 100 form individual optical channels that generate respective images for left and right eyes of a user. Each optical channel includes a display device 110A/110B, a camera 118A/118B, a polarizing beam splitter 120A/120B, and corresponding tube lenses 112A/112B and 112C/112D. In a further embodiment, a polarizer element 114 may be disposed between tube lenses 112A/112B and polarizing beam splitters 120A/120B. Alternatively, polarizer element 114 may include different pieces for respective optical channels.

Although FIG. 1 shows two optical channels for microscope insert 100, one of ordinary skill in the art would recognize that insert 100 may have any number of optical channels, each having a structure similar to those depicted in FIG. 1. When microscope 100 includes two or more optical channels, videos/images generated by the optical channels are configured so as to provide a user with stereoscopic rendering.

In an embodiment, cameras 118A and 118B are digital imaging devices, such as the Point Grey FL3-U3-13S2C-CS manufactured by Point Grey Research. However, a number of different cameras may be used, providing different features, such as a CMOS or CCD based sensor, a global or rolling shutter, and a range of resolutions at about 20 FPS or higher.

In an embodiment, display devices 110A and 110B may be LCOS (Liquid Crystal on Silicon) microdisplay devices, each of which has pixels that can be individually adjusted to match or exceed the brightness of the microscope. Other display technologies may also be used, such as OLED, DLP, T-OLED, MEMS, and LCD-based displays,

Insert 100 also includes a display driver circuit 102 to control display devices 110A and 110B and/or other system elements or features. Display driver circuit 102 may generate video/image data that are suitable for rendering by display devices 110A and 110B,

Insert 100 is connected to a processing unit 108 via standard communication protocols. Processing unit 108 may or may not be disposed within insert 100. Processing unit 108 receives video/image signals from cameras 118A and 118B and sends the video/image signals to driver circuit 102 for rendering the videos/images on display devices 110A and 110B. Processing unit 108 may apply additional processing on videos/images data received from cameras 118A and 118B. For example, processing unit 108 may perform image processing techniques, such as image registration, pattern recognition, image filtering, image enhancement, and the like.

Processing unit 108 may also be connected to other peripherals to collect data to be used by microscope insert 100, to generate visual guidance for navigation during a surgical procedure, or to provide alternative graphical user interfaces on external display devices to supplement the display through microscope insert 100.

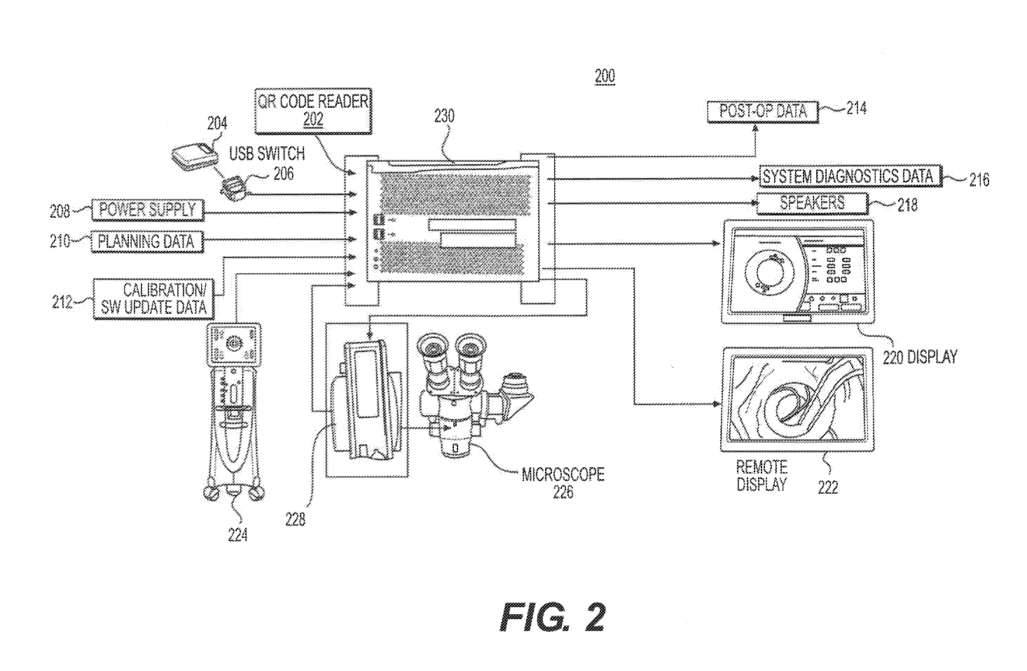

FIG. 2 illustrates a surgical system 200 including a microscope insert 228 according to a further embodiment. Surgical system 200 includes a microscope 226 coupled to microscope insert 228. Microscope insert 228 generally corresponds to microscope insert 100 of FIG. 1. Insert 228 communicates with a processing unit 230, which corresponds to processing unit 108 of FIG. 1.

Microscope 226 receives light or optical signals reflected from an object through its lens system and the polarized beam splitters (e.g., PBS's 120A and 120B), which pass the optical signals to the cameras (e,g., cameras 118A and 118B) of microscope insert 228. The cameras of microscope insert 228 convert the optical signals to digital data representing videos/images of the object and transmit the digital data to processing unit 230.

Processing unit 230 performs image processing on the digital data and sends processed data and relevant commands to the driver circuit (e.g., driver circuit 102) of microscope 226. Based on the processed data and the commands from the driver circuit, display devices (e.g., display devices 110A and 110B) of microscope insert 228 generate optical signals representing processed videos/images of the object and project the optical signals to polarized beam splitters 120A and 120B. Polarized beam splitters 120A and 120B pass the optical signals to the eye pieces of microscope 226 for viewing by a user. The driver circuit may also control, for example, the brightness or contrast of display devices 110A and 110B.

Processing unit 230 may also communicate with additional input devices, such as a QR code reader 202, a foot pedal 204, a USB switch 206, a power supply 208, and one or more external storage devices providing surgical planning data 210 or calibration and software update data 212. Additionally, processing unit 230 may be further connected to a surgical support system 224 that is suitable for the underlying surgery. For example, surgical support system 224 may be the Stellaris system manufactured by Bausch & Lomb Incorporated and suitable for ophthalmic procedures. Surgical support system 224 may collect the demographical and biological data of a patient and provides the data to processing unit 230.

Still additionally, system 200 may include various output devices, such as speakers 218, an external display device 220, and a remote display device 222. External display device 220 and remote display device 222 may be high-resolution monitors that provide additional monitoring capability outside of insert 228. Display devices 220 and 222 may be located in the same operating room as microscope 226 or at a remote location. System 200 may further include one or more storage media for storing post-operation data 214 and system diagnostics data 216. Similarly, other system components shown in FIG. 2 may also be located in different locations and connected to processing unit 230 through, for example, Ethernet, Internet, USB connections, Bluetooth connections, infrared connections, cellular connections, Wi-Fi connections, and the like.

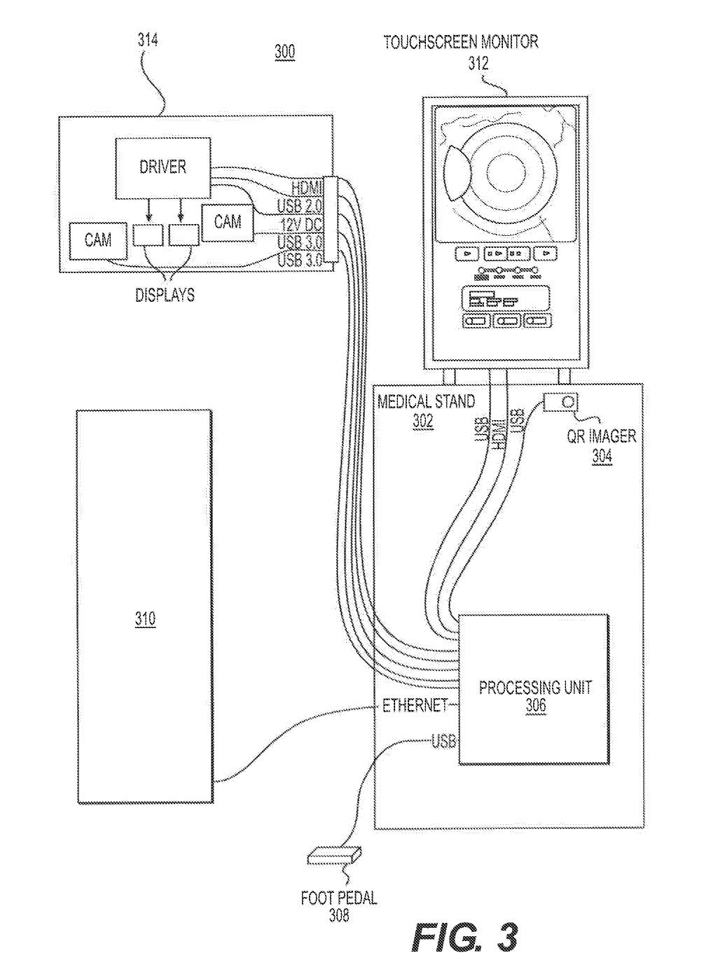

FIG. 3 illustrates a surgical system 300 including a microscope insert 314 according to an alternative embodiment. Microscope insert 314 generally corresponds to microscope insert 100 of FIG. 1 and is configured to generate stereoscopic images as described herein. For example, insert 314 may include two imaging cameras, two display devices, a driver circuit, and other imaging and projection optics for left and right eyes of a user.

System 300 further includes a medical stand 302, an external monitor 312, a foot pedal 308, and a surgical support system 310. Medical stand 302 may include a QR image scanner 304 configured to scan QR codes to provide information encoded in the codes. Medical stand 302 also includes a processing unit 306, which generally corresponds to processing unit 108 of FIG. 1. Processing unit 306 may be included a motherboard with interfaces, such as USB 2.0, USB 3.0, Ethernet, etc. Processing unit 306 may include a central processing unit (CPU) with heat sinks, a RAM, a video card, a power supply, a webcam, etc. Processing unit 306 is connected to other system components through its communication interfaces, such as USB ports, Ethernet ports, Internet ports, HDMI interfaces, etc. For example, processing unit 306 may be connected to microscope insert 314 and external monitor 312 through HDMI interfaces to provide high resolution video/image data to the driver circuit of insert 314 and monitor 312. Alternatively, processing unit 306 may also be connected to insert 314 and monitor 312 through USB ports to provide video/image data and control signals. Processing unit 306 may be connected to the camera of insert 314 through USB ports to receive video/image data from the camera.

Foot pedal 308 and other user input devices may be connected to processing unit 306 through one or more USB ports. Foot pedal 308 may be operated by a user to provide user input during a surgery. For example, when the user presses foot pedal 308, foot pedal 308 may generate an electronic signal. Upon receiving the electronic signal from foot pedal 308, processing unit 306 may control insert 314 accordingly.

For example, when the user presses foot pedal 308, processing unit 306 may control insert 314 to change the videos/images generated by the display devices of insert 314. With each pressing of foot pedal 308, insert 314 may toggle between two sets of videos/images. Alternatively, insert 314 may cycle through a series of videos/images when foot pedal 308 is pressed. Still alternatively, pedal 308 may have a position sensor that generates a position signal indicating a position of pedal 308 when the user partially presses pedal 308. Upon receiving the position signal from pedal 308, processing unit 306 may determine the current position of pedal 308 and control insert 314 accordingly. Processing unit 308 may control insert 314 to generate a different set of videos/images corresponding to each position of pedal 308. For example, when the user presses pedal 308 to a first position, processing unit 306 controls insert 314 to generate a first set of videos/images. When the user presses pedal 308 to a second position, processing unit 306 controls insert 314 to generate a second set of videos/images.

Surgical support system 310 may include an external data source and other surgical systems, such as a Bausch & Lomb Stellaris surgical system. Surgical support system 310 may include biological sensors that collect biological or physiological data of the patient, including, for example, heart rate, blood pressure, electrocardiogram, etc. Surgical support system 310 may further include a database that stores information of the patient, including the patient's medical history and healthcare record. The database may also include information of the underlying surgical procedure such as pre-operation analysis and planning performed by a physician, data collecting during the surgical procedure, and additional procedures recommended for post-operation follow-ups. The database may also include information of the operating physician including his or her identification, association, qualification, etc. Surgical support system 310 may be further connected to additional medical devices (not shown) such as an ultrasound imager, a magnetic resonance imaging device, a computed tomography device, etc., to collect additional image data of the patient.

Processing unit 306 may receive the information and data from surgical support system 310 and controls insert 314 to generate images based on the information and data. For example, processing unit 306 may transmit the additional image data (i.e., ultrasound data, MRI data, CT data, etc.) received from system 310 to the driver circuit of insert 314 and control the driver circuit of insert 314 to render the additional image, through the display devices, along with the microscopic images of the patient provided by the microscope. Processing unit 306 may also generate additional image data representing the biological or physiological data collected from the patient and control insert 314 to render the additional image data through the display devices of insert 314.

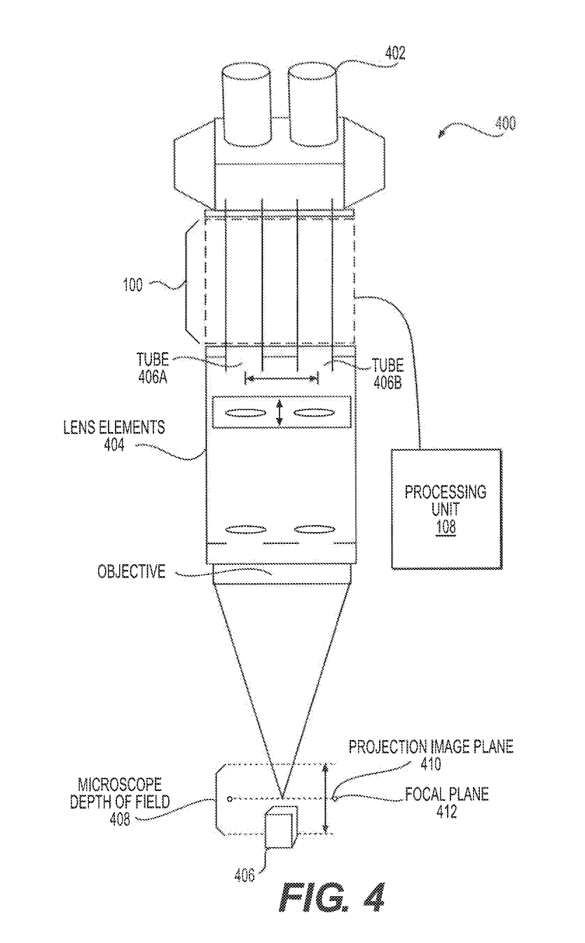

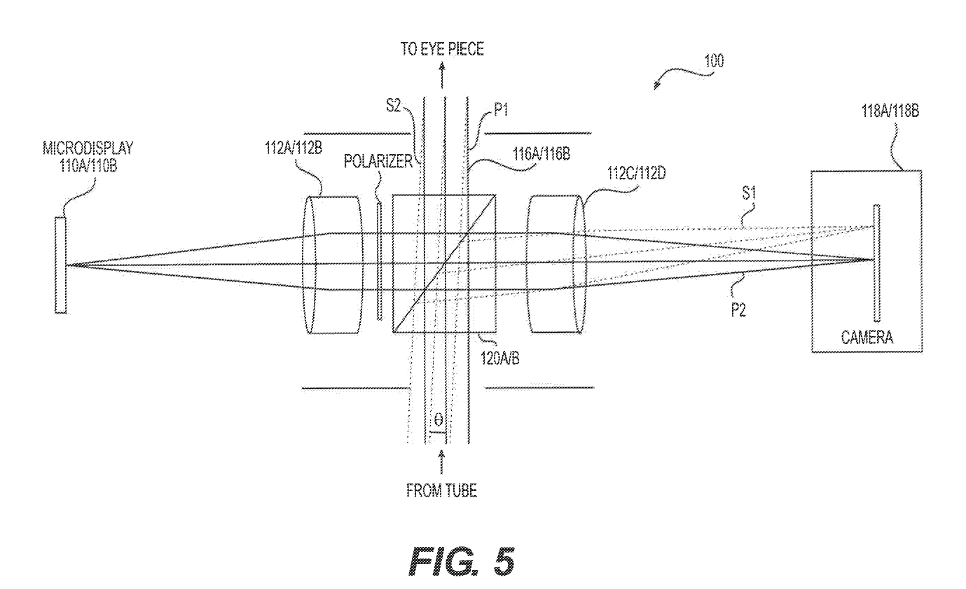

FIGS. 4 and 5 illustrate the operation of a microscope insert according to an embodiment using insert 100 as an example. As shown in FIG. 4, microscope insert 100 may be integrated with a microscope 400 that is suitable for various purposes. In an embodiment, microscope 400 may be a stereoscopic, infinity-corrected, tube microscope. Alternatively, microscope insert 100 may be adapted for use in other microscope layouts and stereoscopic devices known in the art.

Microscope 400 may include a viewing device 402 that allows a user to view images of an object 406 placed under the microscope. Viewing device 402 may be a heads-up device including one or more eye pieces, through which the images of the object are presented to the user. Microscope 400 further includes a set of lens elements 404 that receive light reflected from the object and form microscopic images of the object based on the reflected light. Lens elements 404 transmit the microscopic images of the object to tubes 406A and 406B of microscope 400. Tubes 406A and 406B form light transmission paths (i.e., light paths) that direct the microscopic image of the object toward viewing device 402. The microscopic image may be an analog image in an embodiment.

As further shown in FIGS. 4 and 5, when insert 100 is installed in microscope 400, the polarizing beam splitters 120A and 120B are disposed in the respective light paths between lens elements 404 and viewing device 402 of the microscope, intercepting light coming from respective tubes 406A and 406B. The beam splitters 120A and 120B may also be placed at other locations within the microscope as one of ordinary skill in the art will appreciate. As further described below, beam splitters 120A and 120B may serve two functions in insert 100. First, they may direct a first component of the light signals coming from the object to respective cameras 118A and 118B so that cameras 118A and 118B capture images of the object. Second, they may merge a second component of the light signals coming from the object that is passed through to viewing device 402 with light signals projected from the display devices 110A and 110B.

In particular, in an infinity-corrected tube microscope, for example, light rays passing through the tube are generally parallel, similar to those from a source infinitely far away. Beam splitter 120A/120B splits the light coming up from the object into two portions, directing a first portion (i.e., an S-polarized component S1) towards camera 118A/118B and a second portion (i.e., a P-polarized component P1) towards viewing device 402 of the microscope. Lens 112C/112D between beam splitter 120A/120B and camera 118A/118B is used to focus the S-polarized component S1 exiting beam splitter 120A/120B onto the imaging sensor of camera 118A/118B.

More particularly, polarizing beam splitter 120A/120B receives light signals representing a microscopic image of the object from lens elements 404 through tubes 406A and 406B. Each of polarizing beam splitters 120A and 120B splits incident light signals by allowing one polarized component S1 to reflect and the other polarized component P1 to pass through. The polarized component P1 that passes through beam splitter 120A/120B reaches viewing device 402 and provide the user with the microscopic image of the object for viewing.

The polarized component S1 is reflected by beam splitter 120A/120B toward respective camera 118A/118B through respective tube lens 112C/112D. Camera 118A/118B receives the polarized component S1 reflected from beam splitter 120A/120B and converts the optical signals to electronic image data corresponding to the microscopic image of the object. Camera 118A/118B may then transmit the electronic image data to processing unit 108 for further processing.

Beam splitter 120A/120B operates in a similar manner on the display device side. In particular, display device 110A/110B renders images under the control of the driver circuit and projects light signals corresponding to the images to beam splitter 120A/120B through lens 112A/112B. Lens 112A/112B between beam splitter 120A/120B and respective display device 110A/110B converts the light signals projected from display devices 110A/110B to parallel light rays to match the up-ward parallel light rays coming from tube 406A/406B. Beam splitter 120A/120B splits the incident light signals coming from display devices 110A/110B, reflecting the S-polarized component S2 of the incident light signals originating from display devices 110A/110B and passing through the P-polarized component P2 to camera 118A/118B.

At viewing device 402, the reflected S-polarized component S2 from display devices 110A/110B is then merged or combined with the P-polarized component P1 passed through beam splitter 120A/120B from tube 406A/406B. As a result, the images of the object provided by the P-polarized component P1 and the images from display device 110A/110B provided by the S-polarized component S2 may be simultaneously viewed by the user through viewing device 402. In other words, when viewed through viewing device 402, the images generated by display devices 110A/110B appear as overlaid images on the images of the object formed by lens element 404.

Polarizing element 114 placed between lens 112A/112B and beam splitter 120A/120B is configured to adjust the polarization of those projected parallel rays from lens 112A/112B so as to adjust the ratio of the light component (i.e., the S2 component) reflected by beam splitter 120A/120B to the light component (i.e., the P2 component) passed through to camera 118A/118B. Accordingly, the intensity of the S-polarized component 32 may be adjusted relatively to the intensity of the P-polarized component P2. In an embodiment, the intensity of the S-polarized component S2 may be substantial equal to the P-polarized component P2 so that the light signals projected from display devices 110A/110B are equally split by beam splitter 120A/120B.

Additionally, by adjusting the polarization imposed by polarizing element 114, the intensity of the S-polarized component S2 may also be adjusted relatively to the intensity of the P-polarized component P1. As a result, the images on the display device 110A/110B may be adjusted to be brighter or dimmer with respect to the images of the object when viewed through viewing device 402.

According to a further embodiment, when the P-polarized component P1 and the S-polarized component 32 are combined by beam splitter 120A/120B, the user of microscope 400 may view a combined image including the microscopic image of the object and the overlaid image generated by display device 110A/110B. The optical components of the microscope insert may be adjusted so that the overlaid image may appear at a projection image plane 410 that substantially overlaps the focal plane of microscope 400 and is located within the depth of field 408 of microscope 400.

The microscope insert for a stereoscopic microscope, as shown in FIGS. 1-5, includes a set of imaging and projection hardware for each of the right and left tubes of the microscope so as to generate stereoscopic images. As a result, the insert includes four lenses 112A-112D, lens 112C and 112D configured to focus the images of the object to left and right camera 118A and 118B, and lens 112A and 112B configured to project the images generated by left and right display devices 110A and 110B to beam splitters 120A and 120B. In order to maximize optical efficiency and reduce aberrations, these lenses may be incorporated in a lens set.

In alternative embodiments, the microscope insert may include additional optical components, such as mirrors, prisms, or lenses, in the optical paths between the beam splitters and the cameras or between the beam splitter and the display devices to modify the directions of the light rays. The modified light rays may allow the optical components of the insert to be more freely arranged or repositioned so as to fit into a desired mechanical or industrial form.

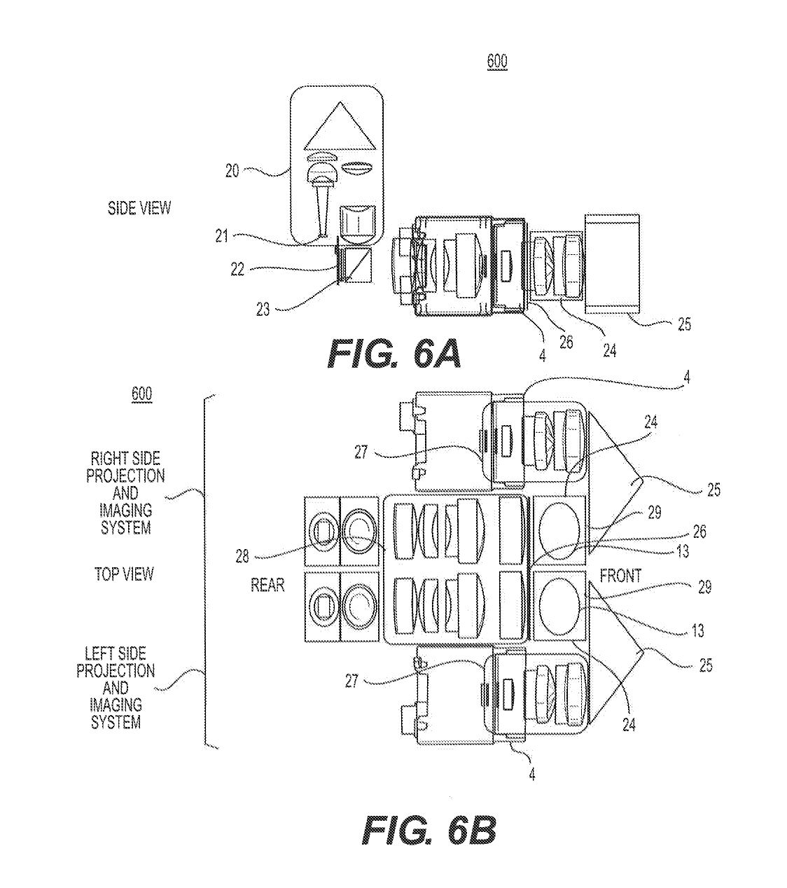

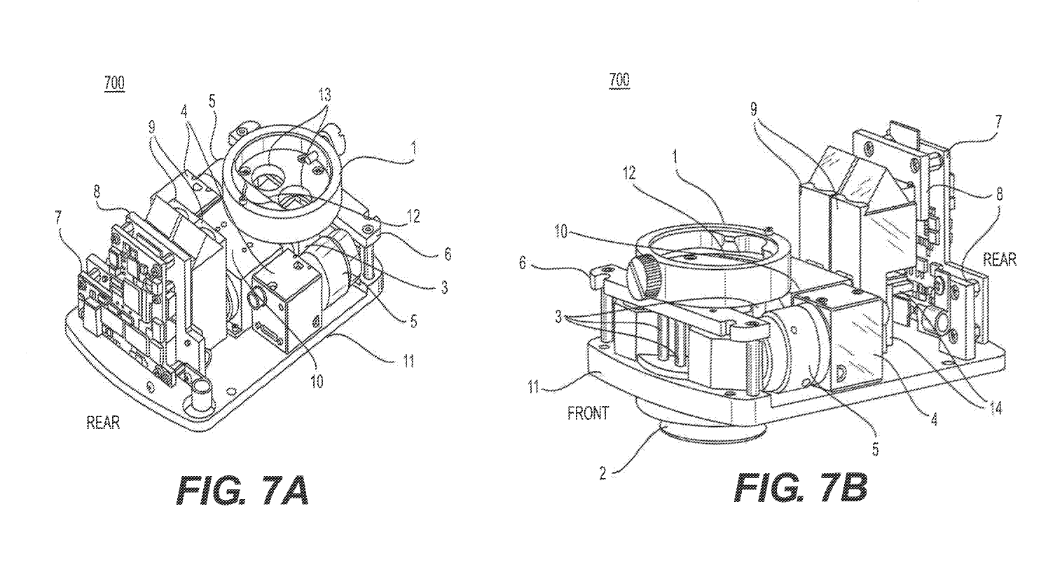

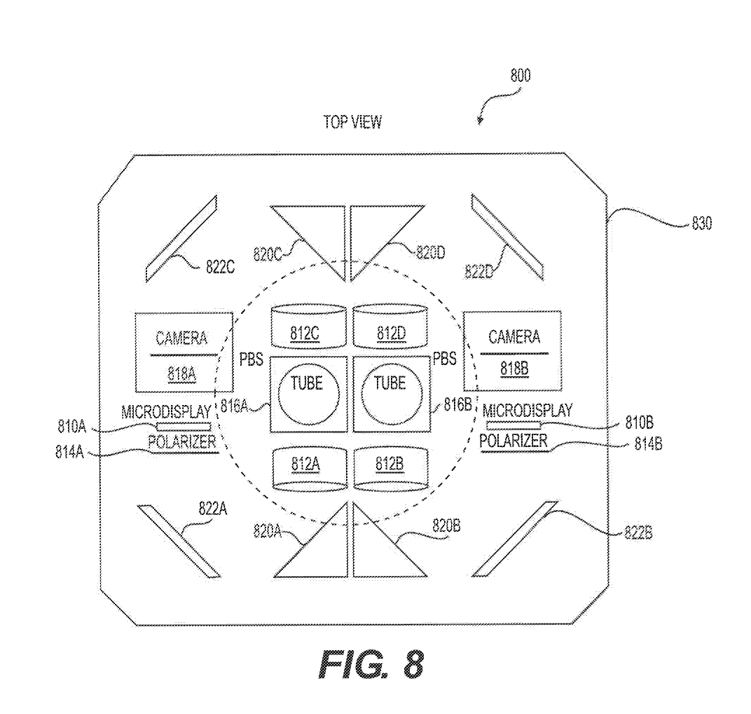

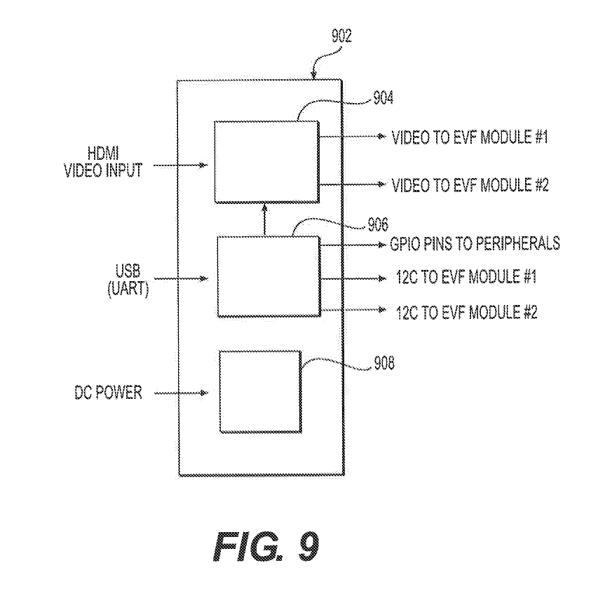

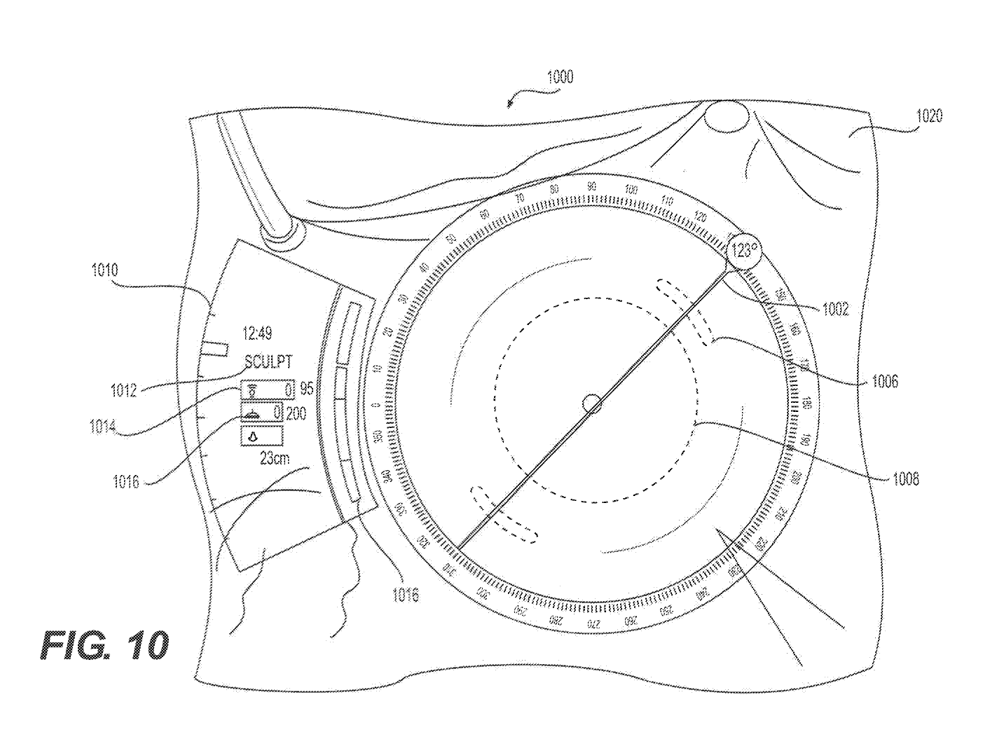

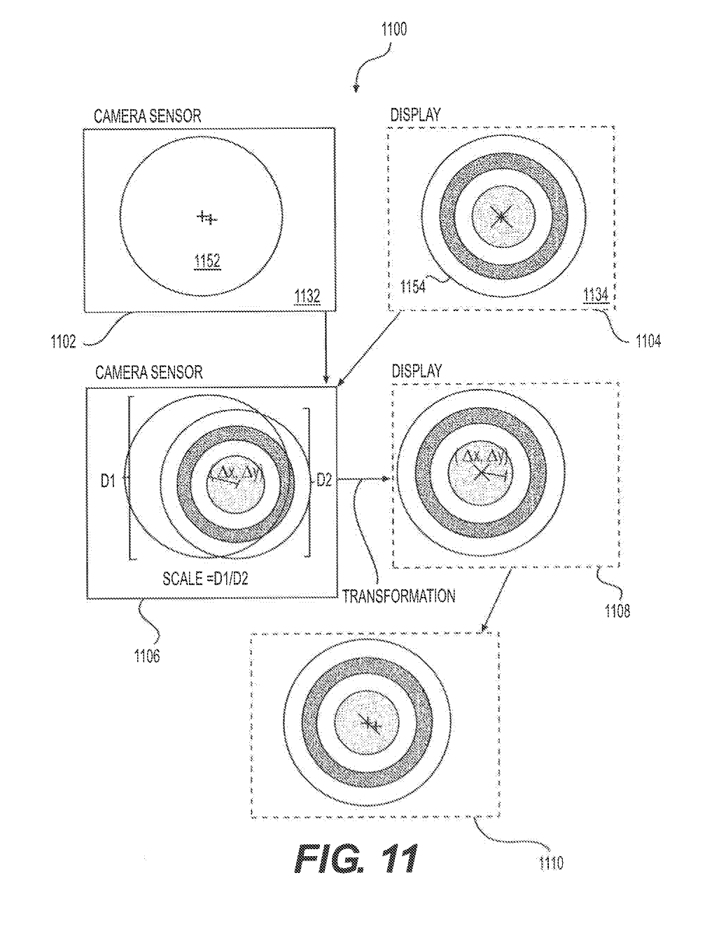

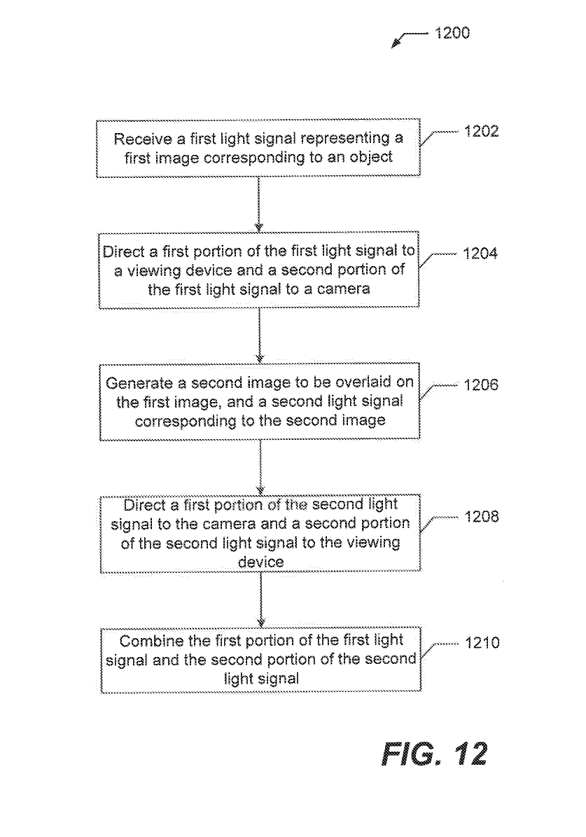

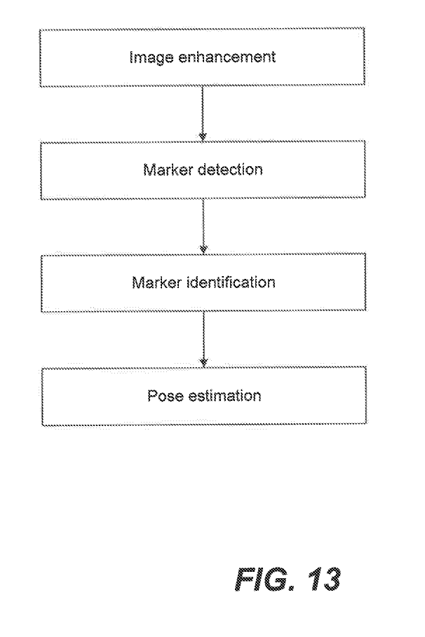

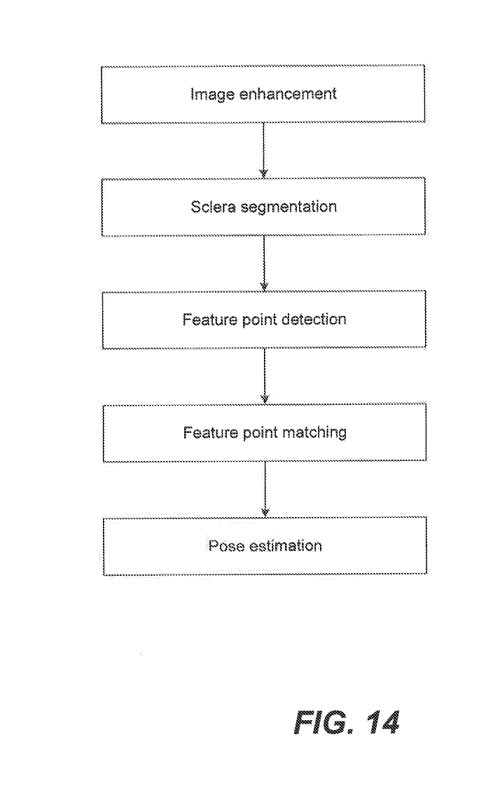

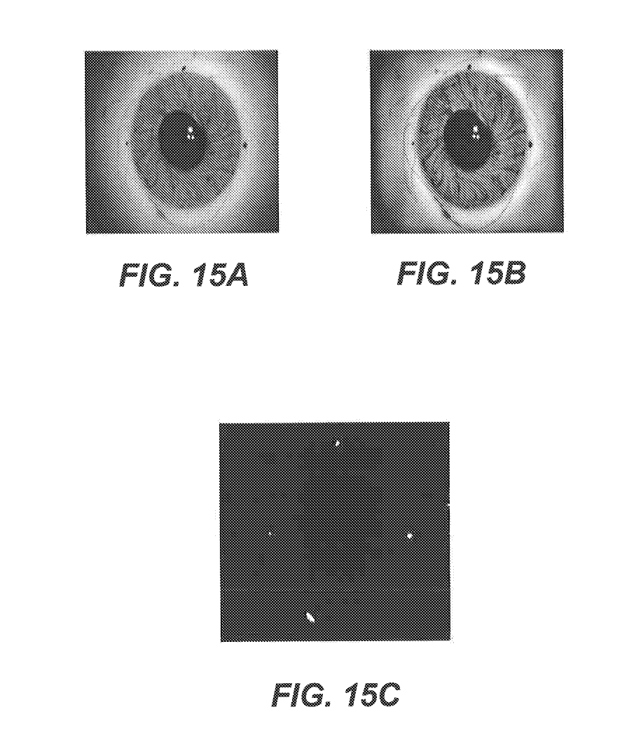

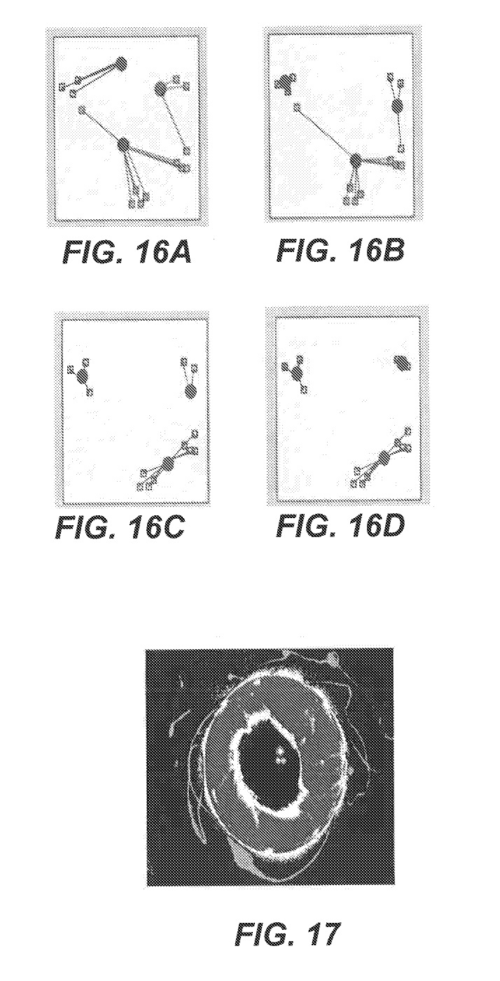

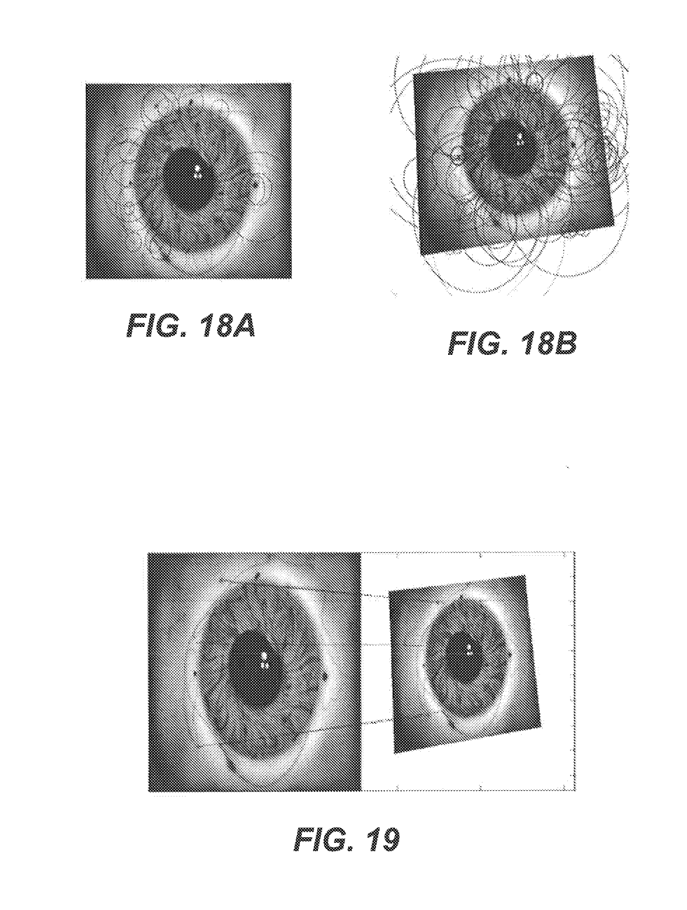

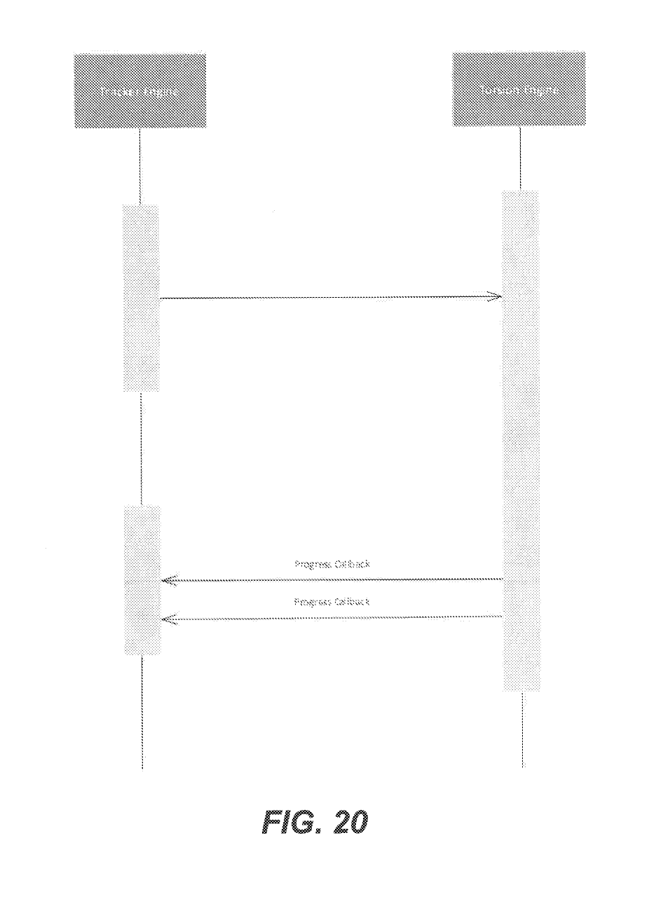

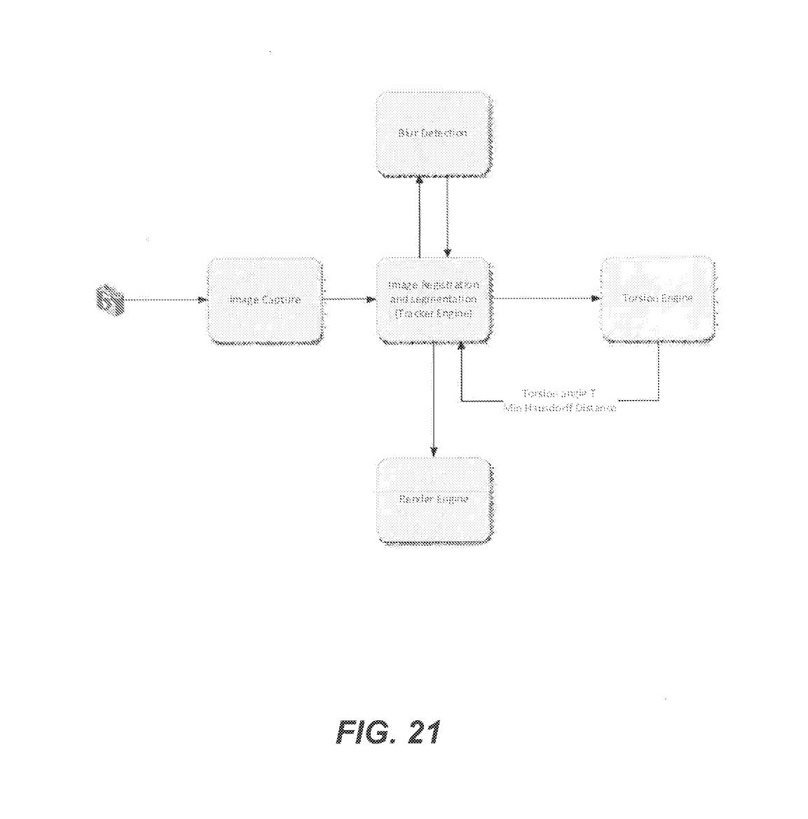

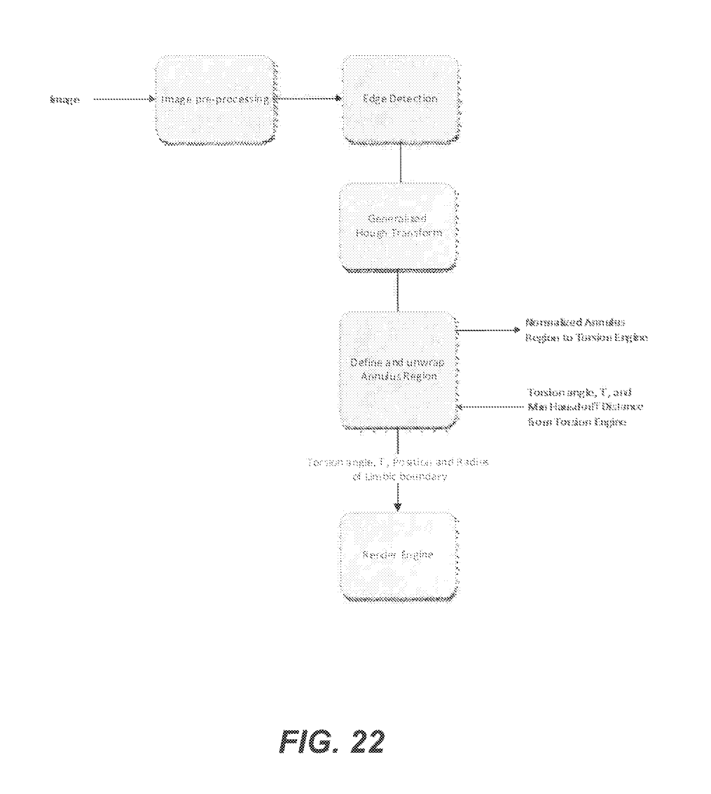

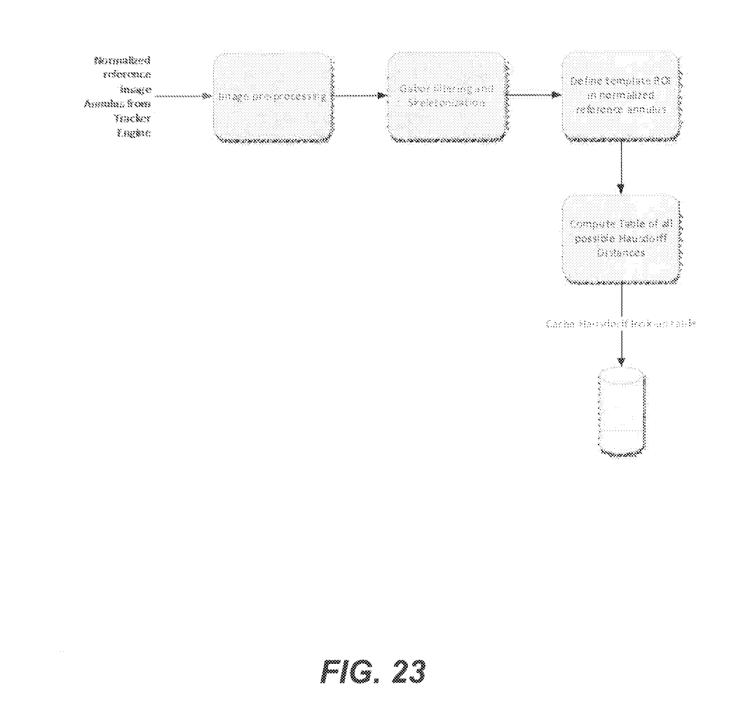

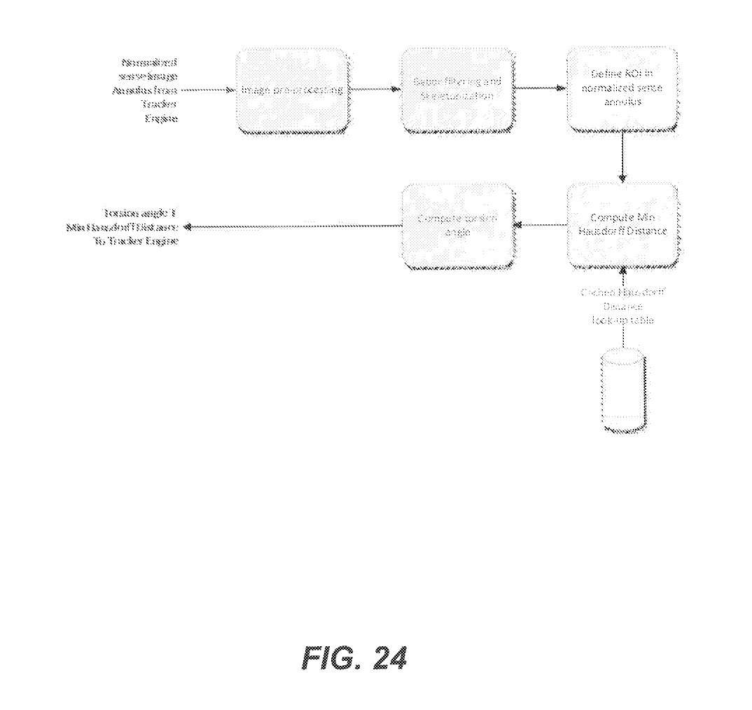

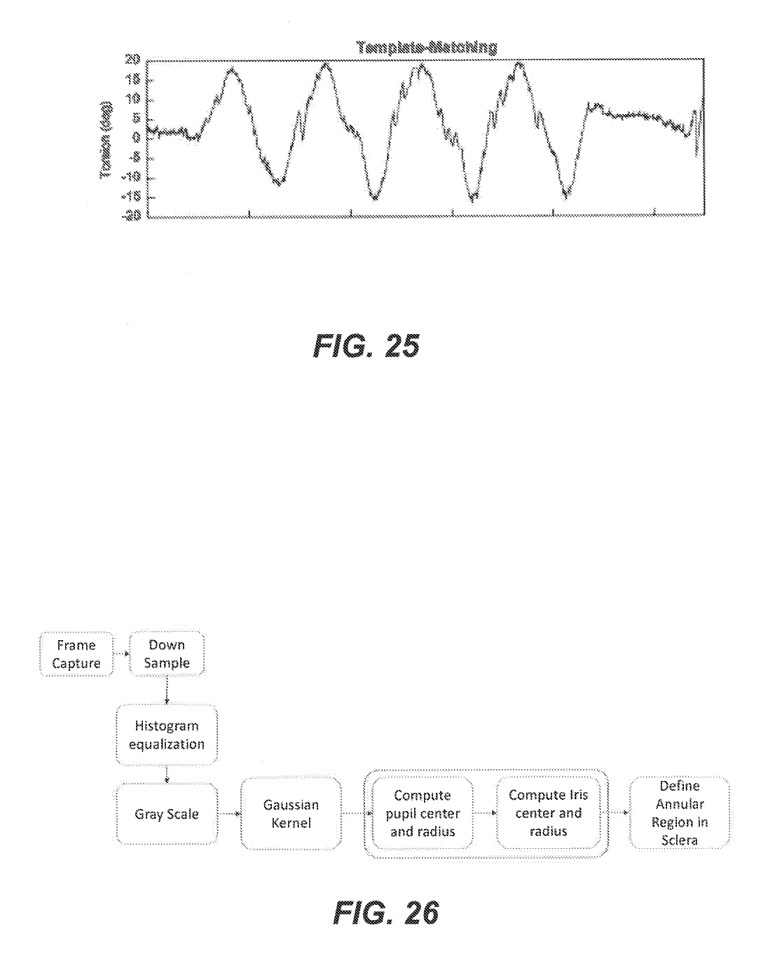

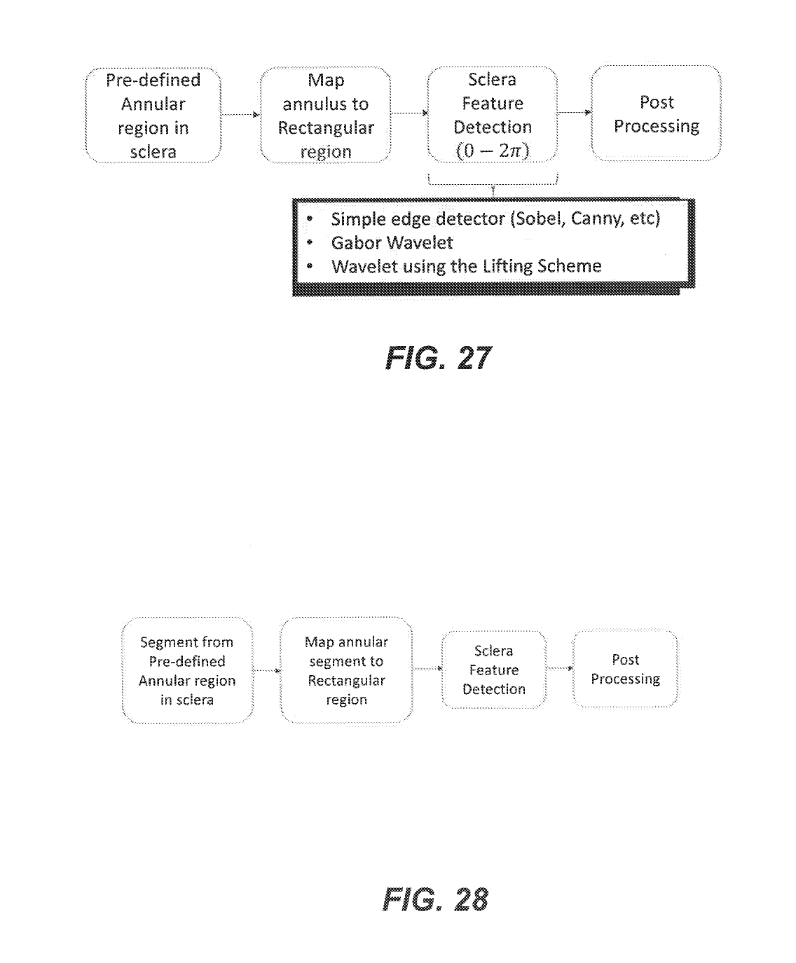

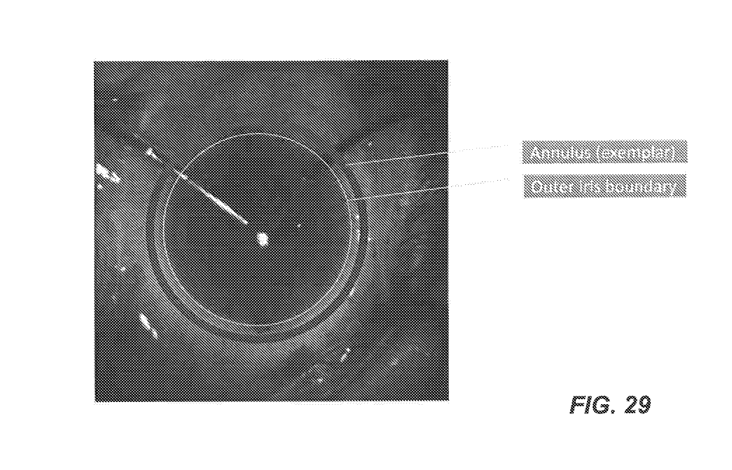

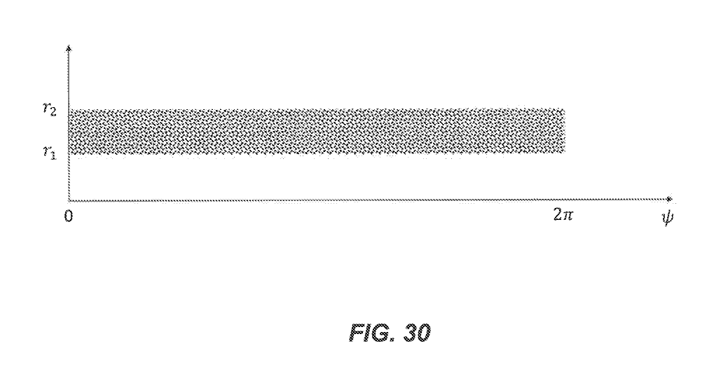

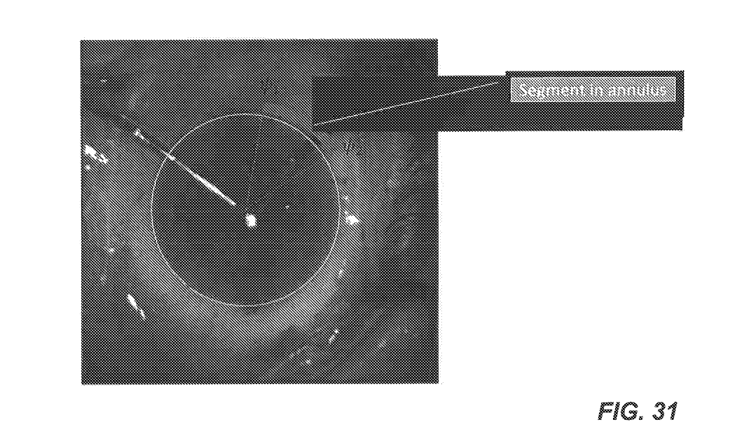

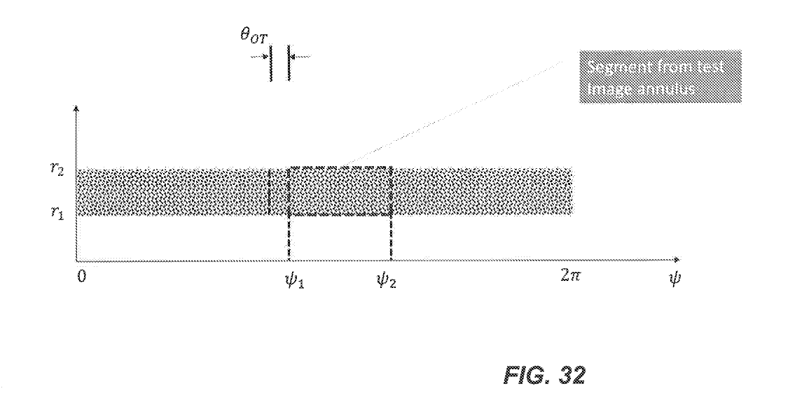

FIGS. 6A and 6B illustrate an embodiment of a microscope insert 600 including additional optical components to steer light rays. FIGS. 6A and 6B shows, respectively, a side view and a top view of major optical elements of microscope insert 600. Microscope insert 600 includes two optical channels for rendering images, respectively, for left and right eyes of the user. Although only one optical channel is described here, one of ordinary skill in the art will appreciate that the optical channels include similar elements and operate in similar manor.

Each optical channel of microscope insert 600 includes a polarizing beam splitter 624 disposed in the corresponding light pathway of the microscope and coupled to the tube of the microscope, from which light reflected by an object enters microscope insert 600. A portion (i.e., the S-polarized component S1) of the incident light is diverted to a turning prism 625, which directs the S1 component through imaging lenses 627 on to a camera 604.

The other portion (i.e., the P-polarized component P1) of the incident light passes through a polarizing beam splitter 624 and reaches the eyepiece of the microscope to provide a microscopic image of the object that is placed under the microscope. In an additional embodiment, beam spatter 624 may include a polarizer element configured to adjust the ratio of the light component diverted to camera 604 to the light component passed through to the eyepiece. The ratio may be, for example, 1:1, 1:2, 1:3, or other desired value.

The images generated by the processing unit and to be overlaid on the microscopic images of the object are rendered by a projection LCOS display panel 622 illuminated by an ROB LED light source 621. The S-polarized light component S2 of the light generated by LED light source 621 is passed through a set of display illumination optics 620 including illumination lenses and a turning prism. From illumination optics 620, the S-polarized light component S2 is reflected at the hypotenuse of a polarizing beam splitter 623 to LCOS display panel 622. LCOS display panel 622 acts as an active polarizer. The P-polarized light component P2 passes through a projection lens module 628 and a polarizing wave plate 626 to tube polarizing beam splitter 624. The P-polarized light component P2 is then directed to camera 604 by tube polarizing beam splitter 624 and steering prism 625. The S-polarized light component S2 is diverted and reflected by tube polarizing beam splitter 624 to the eyepiece of the microscope, which then visualizes the microscopic images of the object and the images generated by display panel 622. When viewed through the eyepiece, the images generated by display panel 622 are overlaid on the microscopic images of the object.

Alternatively, polarizing wave plate 626 may be omitted. Accordingly, the light from LCOS display panel 622 passes through tube polarizing beam splitter 624 without being reflected to the eye piece. Instead, the light from LCOS display panel 622 is directed to turning prism 625 and, in turn to, imaging lens 627 and camera 604. The benefit of this configuration is that wave plate 626 can be removed to perform a calibration between display panel 622 and camera 604. Based on calibration, the system may confirm that images generated by display panel 622 are aligned to the image space being measured by camera 604.

FIGS. 7A and 7B illustrate an embodiment of a microscope insert 700 that is similar to microscope insert 600 described above. The components of microscope insert 700 are packaged and assembled on a base plate 711 so that microscope insert 700 is ready to be installed on a microscope. In particular, insert 700 includes one or more optical channels, each including components similar to those of insert 600 illustrated in FIGS. 6A and 6B.

Each optical channel includes a camera 704 disposed in a camera housing affixed to base plate 711, a set of imaging lenses disposed in a lens tube 705, an imaging steering prism secured to base plate by prism bracket 706, a set of illumination optics disposed in an illumination optics housing 709, a set of projection lenses disposed in a lens tube 710. A focus mechanism is provided in imaging lens tube 705 and allows for fine adjustment of the relative position of the imaging lenses therein, for focusing. Likewise, a focus mechanism is also provided in display lens tube 710 and allows for fine adjustment of the position of the projection lenses for focusing.

Each optical channel further includes an RGB LED light source and a display panel mounted to base plate 711 through a display and RGB LED mounting bracket 714. Microscope insert 700 further includes a driver circuit board 707 mounted to base plate 711 through a driver board bracket 708.

Microscope insert 700 further includes mounting components for mounting onto a microscope. For example, insert 700 includes a top mount 701 that may be coupled to the eyepieces of the microscope. Top mount 701 may include features that allow the eyepieces to be secured thereon. Top mount 701 is secured to base plate 701 through one or more top mount braces. Top mount 701 includes one or more microscope tube openings that allow light to pass through from the polarizing beam splitters to the eye pieces of the microscope. Top mount 701 further includes a wave plate slot 712 for disposing and securing the wave plate. The wave plate may be easily inserted into wave plate slot or removed therefrom as desired. Microscope insert 700 further includes a bottom mount flange 702 that may be coupled and secured to the microscope tube within the body of the microscope.

FIG. 8 illustrates a microscope insert 800 according to another embodiment. In this embodiment, light reflected from the object under the microscope (not shown) is directed from the tubes (e.g., 406A and 406B of FIG. 4) to respective cameras 818A and 818B by a group of reflective mirrors and prisms 820C, 820D, 822C, and 822D. Similarly, the images generated by display devices 810A and 810B are projected back to beam splitters 816A and 816B by another group of mirrors and prisms 820A, 8208, 822A, and 822B. The arrangement in this embodiment allows the components to be disposed on a relatively small base plate that has a relatively small footprint, thereby easing integration in a variety of microscopic systems.

As further shown in FIG. 8, a polarizer element 814A/814B may be disposed in the light path between display device 810A/810B and beam splitter 816A/816B and is used to vary the amount of light passed through to camera 818A/818B from display device 810A/810B. Polarizing element 814A/814B may be a set of polarizers, wave plates, or variable retarders, depending on the output polarization of display devices 810A and 810B. In an embodiment, display device 810A/810B outputs an S-polarized component, which is then rotated by a ½-lambda wave plate in polarizing element 814A/814B so as to be reflected upwardly to the eyepiece for viewing by the user.

The microscope inserts disclosed herein may create a stereoscopic image. In particular, the inserts may create separate images for the left and right eyes of the user. The images are shifted with respect to each other to provide the perception of different convergence, resulting in stereoscopic rendering.

FIG. 9 is a schematic diagram of a display driver circuit 900 according to an embodiment. Display driver circuit 900 generally corresponds to driver circuit 102 of FIG. 1. Driver circuit 900 provides communication interfaces between processing unit 108 and display devices 110A and 110B. The functions of driver circuit 900 may include, for example:
Communicating customized resolution HDMI video signals from processing unit 108 to display devices 110A and 110B;
Generating image frames of a desired resolution (i.e., 1976×960), including a side by side (SBS) layout of the left and right images to be displayed to the user;
Using line phasing to split the SBS image frames into left and right image signals;
Directing the image data to each display device 110A/110B; and
Providing a USB interface for communication with processing unit 108, which supports, for example, firmware updates, control of brightness, gamma, color channel gain of each display device, display focus, and status indication (i.e. power indication, insignia illumination, etc.).

According to an embodiment, processing unit 108 analyzes image data provided by cameras 118A and 118B and provides inputs to display driver circuit 102 for generating overlaid images through the display devices 110A and 110B. For example, processing unit 108 may analyze the image data for registration, tracking, or modeling the object under the microscope. Information derived from the analysis of the image data may then be used to generate and adjust the overlaid images generated by display devices 110A and 110B.

In a further embodiment, the microscope insert disclosed herein may be integrated in a microscope for ophthalmic procedures, such as cataract surgery. The microscope insert may generate images representing surgery-related information to assist a surgeon to navigate during a cataract surgery. The images may be displayed to the user overlaid with the real-time microscopic image of the patient's eye. As a result, the surgeon is able to simultaneously view the image of the eye and the overlaid images through the microscope.

FIG. 10 illustrates an exemplary composite image 1000 rendered by a microscope having a microscope insert described herein, according to an embodiment. Image 1000 includes a real-time microscopic image 1020 of a patient's eye as viewed through the microscope and images generated by the microscope insert overlaid on the real-time eye images. Microscopic image 1020 of the patient's eye may be an analog image formed by the zoom lens elements of the microscope. The overlaid images generated by the microscope insert include graphical representations of information related to the surgical procedure. The overlaid images may include prompts or instructions to guide the surgeon during the surgery.

For example, the overlaid images may include image features indicating an axis of interest 1002 and incision points 1006 and 1008 to guide the surgeon to carry out incision and placement of the artificial lens. The overlaid images may also present information including parameters related to the surgery, such as the current operation stage 1012, ultrasound power 1014, vacuum suction 1016, current time, and the like. The information may be presented in an image area 1010 near the area of operation. Image area 1010 may have a shape that generally conforms to the shape of the patient's eye. The processing unit of the microscope insert is configured to track and determine the position, size, and rotation of the patient's eye as it is viewed through the microscope and adjust the position, size, and orientation of the overlaid images accordingly so that the overlaid images remain registered with the patient's eye.

The microscope insert described here may also receive external data from external data sources and user inputs from user input devices during a surgical procedure, and adjust the overlaid images accordingly. For example, during a cataract surgery, the processing unit may receive, from the external data source, demographic information, bio-information, and medical history of the patient. The external data source may include a monitoring system that monitors status of surgical equipment or status of the patient, such as heart rate, respiratory rate, blood pressure, eye pressure, and the like, during the surgery. The processing unit may receive, from the monitoring system, the external data including real-time information representing the status of the patient and the equipment and presenting the external data as part of the overlaid image displayed to the operating surgeon through the microscope insert.

Additionally, the processing unit may receive user inputs from the surgeon through the input devices, such as a joy stick, a foot pedal, a keyboard, a mouse, etc. The user inputs may instruct the processing unit to adjust the information displayed in the overlaid images. For example, based on the user inputs, the processing unit may select portions of the external data for display as part of the overlaid images.

The processing unit may also display prompts or navigation instructions related to the surgical procedure according to the user inputs. For example, when the surgeon completes a step of a surgical procedure and presses the foot pedal, the processing unit may control microscope insert to modify the overlaid images so as to display prompts or instructions for the next step. The prompts or instructions may include text or graphical information indicating the next step and may further include data or parameters relevant to the next step.

The processing unit may also control the microscope insert to generate a warning to alert the surgeon if there are abnormalities during a surgical procedure. The warning may be a visual representation such as a warning sign generated by the display devices as part of the overlaid image. The warning may also be other visual, audio, or haptic feedback, such as a warning sound or a vibration.

During the operation of the microscope insert, the field of view provided by the display device of the insert may be different from the field of view of the microscope. FIG. 11 illustrates a process 1100 for correcting the field of view provided by the display devices and matching it with the field of view of the microscope.

According to process 1100, at step 1102, the microscope generates a microscopic image 1132 having a field of view 1152. At step 1104, the microscope insert generates an overlaid image 1134 having a field of view 1154. In an embodiment, fields of view 1152 and 154 may each have a circular shape. Field of view 1152 may have a diameter D1, and field of view 1154 may have a diameter D2.

At step 1106, overlaid image 1134 generated by the microscope insert and microscopic image 1132 generated by the microscope are displayed to the user through the eyepiece. When viewed through the eyepiece, microscopic image 1132 and overlaid image 1134 are combined or overlaid. However, due to mismatch between the fields of view of the two images, image features of overlaid image 1134 may obscure important image features of microscopic image 1132 or may appear to be disproportional to the image features of microscopic image 1132.

In order to align the fields of view of the two images, overlaid image 1134 must be adjusted according to the field of view of microscopic image 1132. As discussed above with reference to FIG. 5, polarization imposed by polarizing element 114 on light signals projected by display device 110A/110B allows a portion (i.e., the P-polarized component P2) of the light signals to pass through polarizing beam splitter 120A/120B. The passed-through light from display device 110A/110B is received by camera 118A/118B, which captures overlaid image 1134. On the other hand, camera 118A/118B receives light (i.e., the S-polarized component S1) from the object, which is reflected by beam splitter 120A/120B, and captures microscopic image 1132 generated by the microscope. The processing unit (i.e., processing unit 108 of FIG. 1) then compare overlaid image 1134 with microscopic image 1132 to determine image transformations necessary to match field of view 1154 of overlaid image 1134 with field of view 1152 of microscopic image 1132.

At step 1108, the processing unit then applies the image transformations to overlaid image 1134 generated by the display device and control the display device to generate an adjusted overlaid image 1138. As a result, the field of view provided by the display device is properly aligned with the field of view of the microscope at step 1110.

Process 1100 may be used to correct any optical misalignment during manufacturing or slight damages from handling. The image transformations used by the processing unit may be affine transformations. Typical transformations may include translation, scaling, skewing, rotation, and the like. For example, the processor unit may determine a scaling factor for scaling overlaid image 1134 based on a ratio between the diameter D1 of field of view 1152 and the diameter D2 of field of view 1154. The processor unit may also determine translation parameters (Δx and Δy) necessary to align the microscopic image and the overlaid image based on the distance between the circular centers of fields of view 1152 and 1154. Using process 1100, the microscope insert may provide more precisely placed overlaid images over the microscopic images when viewed through the eyepiece of the microscope.

According to additional embodiments, the processing unit may monitor changes in the field of view of the microscopic image (i.e., based on the S-polarized component S1) during operation and adjust the overlaid image in such a way to track or follow the field of view of the microscopic image. Alternatively, the processing unit may track an anatomical feature of the patient under the microscope and adjust the field of view of the overlaid image to follow the anatomical feature.

According to another embodiment, the camera (i.e., camera 118A/118B of FIG. 1) is configured such that field of view 1152 of the microscope is entirely captured by the camera sensor. Similarly, the overlaid image generated by the display device (i.e., display device 110A/110B) is configured to cover entirely field of view 1152 of the microscope. The camera sensor and the display device are configured to provide oversampling so as to provide sufficient resolutions over the image area that covers the field of view of the microscope.

FIG. 12 illustrates a process 1200 for generating an overlaid image over a microscopic image, according to an embodiment. Process 1200 may be implemented on the microscope insert (i.e., microscope insert 100) disclosed herein.

According to process 1200, at step 1202, the microscope insert receives a first light signal from a microscope (i.e., microscope 400). The first light signal represents a first image corresponding to an object (i.e., object 406) placed under the microscope. As shown in FIGS. 4 and 5, the first light signal may be received from the zoom lens elements of the microscope through the tube within the body of the microscope. The first image may be an analog microscopic image of the object.

At step 1204, the microscope insert directs a first portion (i.e., the P-polarized component P1) of the first light signal to a viewing device (i.e., viewing device 402) and a second portion (i.e., the S-polarized component S1) of the first light signal to a camera (i.e., camera 118A/118B). More particularly, the first light signal may be split by the polarizing beam splitter (i.e., PBS 120A/120B) of the microscope insert into the first portion and the second portion. The polarizing beam splitter may be configured to allow the first portion of the first light signal to pass through to the viewing device and reflect the second portion of the first light signal to the camera within the microscope insert. The microscope insert may further include a tube lens (i.e., lens 112C/112D) to focus the second portion of the first light signal onto the camera sensor and/or additional light steering components (i.e., mirrors and prisms) to direct or redirect the second portion of the first light signal to the location of the camera.

At step 1206, a display device (i.e., display device 110A/110B) of the microscope insert generates a second image to be overlaid on the first image. The second image (i.e., the overlaid image) includes graphical representations indicating information relevant to the object. For example, when the object is a patient's eye and a surgical procedure (i.e., a cataract surgery) is carried out on the object, the second image may include, for example, prompts, instructions, parameters, and data relevant to the underlying surgical procedure. By displaying the second image, the display device produces a second light signal representing the second image.

At step 120B, the microscope insert directs a first portion (i.e., the P-polarized component P2) of the second light signal to the camera and a second portion (i.e., the S-polarized component S2) of the second light signal to the viewing device. The second light signal may be split again by the polarizing beam splitter into the first portion and the second portion. The polarizing beam splitter may allow the first portion to pass through to the camera and reflect the second portion to the viewing device. The microscope insert may further include a tube lens (i.e., lens 112A/112B) between the display device and the polarizing beam splitter to alter (i.e., expand) the second light signal projected by the display device. The microscope insert may also include additional light steering components (i.e., mirrors and prisms) to direct the second light signal from the display device to the location of the polarizing beam splitter. The microscope insert may also include a polarizer element (i.e., polarizer element 114) between the display device and the polarizing beam splitter. The polarizer element may impose polarization on the second light signal so as to adjust the ratio between the first portion of the second light signal, which is passed through to the camera, and the second portion of the second light signal, which is reflected to the viewing device.

At step 1210, the first portion of the first light signal and the second portion of the second light signal are combined to form a composite image, including the first image corresponding to the object and the second image generated by the display device. The second image, when viewed through the viewing device, is rendered over the first image. As a result, the user of the microscope (i.e., the surgeon) may simultaneously view the first image (i.e., the microscopic image of the patient's eye) and the second image (i.e., the overlaid image) through the viewing device (i.e., the eyepiece) of the microscope.

Additionally, at step 1210, the microscope insert may detect any mismatch between a field of the view of the first image and a field of view of the second image. The microscope insert may detect the mismatch based on the second portion of the first light signal and the first portion of the second light signal received by the camera. If there is a mismatch, the microscope insert may adjust the second image according to the image transformations described herein so as to match the field of view of the second image with the field of view of the first image.

According to an embodiment, during operation, a microscope insert may apply image registration to the images of the object viewed under a microscope. Based on the registration, the processing unit of the microscope insert may render graphical elements, such as tags, labels, and the like, through the display device, providing instructions, prompts, or other surgery-related information to the operating surgeon. When the surgeon views the object through the eyepiece of the microscope, the graphical elements are overlaid on the images of the object. The overlaid graphical elements may identity and track anatomical features that are of interest and are spatially associated with the identified anatomical features, thereby providing the surgeon with visual guidance and facilitating navigation through the surgical site.

According to some embodiments, the processing unit may be configured to perform two-dimensional (2D) or three-dimensional (3D) registration and tracking. In one embodiment, images generated by the cameras may be analyzed by the processing unit independently to provide 2D registration and tracking. Alternatively, the images generated by the cameras may be analyzed together to provide a 3D registration to a known or assumed model. The disclosed system provides the benefits of improved 3D registration by using two cameras for the 3D registration and two display devices for the 3D overlays. For example, the 3D registration is significantly improved using two cameras, compared with existing systems with one camera, and allows for improved registration and tracking, particularly for anatomical features that are at different depths within an operative site and move with respect to each other.

The tracking and registration of the movements of an object, such as a patient's eye, may be performed based on fiducial markers placed on the object or anatomical features of the object. FIG. 13 illustrates an exemplary process 1300 for the marker-based registration. As shown in FIG. 13, at step 1302, the processing unit of the microscope insert applies image enhancement to the images of the object captured by the camera. The image enhancement may be any known techniques such as digital filtering, sharpening, and the like.

At step 1304, the processing unit may detect and identify fiducial markers disposed on the object. The fiducial markers may be detected based on spatial or spectral analysis of the images of the object. Alternatively, the fiducial markers may be determined based on a predetermined shape or color. The processing unit may further identify the fiducial markers and associate the fiducial markers with respective identifications.

At step 1306, the processing unit may perform pose estimation. For example, the processing unit may further perform registration on the images of the object based on the identified markers. For example, the processing unit may determine a movement or orientation of the object based on the identified marker and calculate a coordinate transformation corresponding to the movement or orientation. The transformation mathematically represents translations, rotations, or other affine transformations of the object. In addition, the processing unit may adjust the graphical elements of the overlaid images generated by the display device according to the registration. The processing unit may apply the coordinate transformation to the graphical elements so that the graphical elements experience similar translations, rotations, and the like. In an embodiment, the registration is carried out in real-time when the images of the object are captured by the camera.

FIG. 14 illustrates an exemplary process 1400 for the anatomical feature-based registration. According to process 1400, at step 1402, the processing unit performs image enhancement on the images of the object captured by the camera, similar to step 1302 discussed above. At step 1404, the processing unit performs image segmentation on the enhanced images of the object. The image segmentation may be performed on an entire image or on regions of the image. The image segmentation may be based on any known techniques, such as, the clustering techniques, histogram techniques, and thresholding techniques. During the image segmentation, image pixels are grouped according to the image features, to which they belong.

At step 1406, the image features are detected based on the segmented images of the object. Individual image features that are of interest may be extracted from the segmented images. The detected image features may correspond to known anatomical structures of the object. The detected image features may then be matched with the known anatomical structures based on, for example, their shapes, sizes, locations, colors, and the like.

At step 1408, the processing unit may perform pose estimation similar to step 1306. For example, the processing unit may use a random sampling technique to calculate the pose of the object based on the detected image features. The processing unit may determine a coordinate transformation corresponding to the pose of the object. In addition, the processing unit may apply the coordinate transformation to the image elements of the overlaid image generated by the display device. As a result, the overlaid image tracks the anatomical features of the object when viewed through the microscope.

In either the fiducial marker-based process or the anatomical feature-based process discussed above, a 2D or 3D registration may be achieved. In two-dimensional registration, the processing unit determines, based on the processed image, a set of coordinate transformation data including, for example, X, Y, R, and Theta. X and Y represent the position, in pixel space, of the center of the patient's eye. R represents the radius, in pixel space, of the limbus of the patient's eye. Theta represents an angle of rotation of the patient's eye.

In three-dimensional registration, the processing unit may use information from the images captured by the left and right cameras to solve for the coordinate transformation data of the object on six degrees of freedom. Additionally, the processing unit may also use a hybrid registration technique that combines elements of the fiducial marker-based registration and the anatomical feature-based registration to determine the position and orientation of the eye of a patient.

As shown in FIGS. 13 and 14, in both the fiducial marker-based method and the anatomical feature-based method, the processing unit first performs an image enhancement on the images of the object acquired by the camera. A range of image enhancement techniques may be used. For example, as shown in FIGS. 15A-15C, the processing unit may apply a histogram equalization technique to an image of an eye acquired by the camera (FIG. 15A). A known contrast-limited adaptive histogram equalization may be used to equalize the histogram by broadening the range of the contract of image intensities. In a further embodiment, the processing unit first converts the image into an L-a-b color space representation, in order for the histogram equalization to be performed on the luminosity channel of the converted image. FIG. 15B shows a resulting enhanced image after the histogram equalization.

Following the contrast enhancement, the processing unit extracts the fiducial markers by segmenting the enhanced image. For example, in the fiducial marker-based registration, the processing unit may select a saturation channel in an HSV color space representation and apply binary thresholding to the enhanced image. FIG. 15C shows a binary image produced by binary thresholding. The binary image may be further digitally filtered by the processing unit to eliminate those regions that have a smaller number of pixels than a preset value. The further filtering may eliminate those regions that do not correspond to any markers. The processing unit may also remove highly eccentric regions having a large difference between major and minor diameters. The centroid of the remaining pixel clusters may then be computed and stored as the correct locations of the fiducial markers.

According to another embodiment, in the anatomical feature-based registration for an eye, the processing unit may further perform a contrast-limited adaptive histogram equalization (CLAHE) on the image acquired by the camera to enhance the contrast in the image. The processing unit may then apply a Gaussian filtering on the enhanced image and then segment the filtered image into regions based on color similarities in the a-b space of the L-a-b color space representation. The processing unit may then apply a K-means clustering technique known in the art, as shown in FIGS. 16A-16D, to achieve color segmentation on a real-time scale. The K-means clustering technique applies vector quantization on the image pixels and partitions the pixels into k clusters, in which each pixel belongs to the cluster with the nearest mean values. The K-means clustering technique starts from an initial guess (FIG. 16A) and gradually refines the clusters (FIGS. 16B-16D) so that the image pixels are properly grouped in the feature space.

The resulting image may then be thresholded to define a binary mask, which may later be used to filter out or remove feature points inside the iris of the eye as shown in FIG. 17. This is desired because the size and shape of the iris may change throughout the surgery, making any feature points within the iris unsuitable for tracking.

As further shown in FIGS. 18A, 18B, and 19, feature points of an eye along with their describing geometries, known as feature descriptors, are identified in a reference image (FIG. 18A) and a test image (FIG. 18B). A known real-time feature point detection technique or scale-invariant technique may be used to classify these features. These techniques include, for example, SIFT (Scale-Invariant Feature Transform), SURF (Speeded-up Robust Features), STAR, FAST, GFTT (Good Features to Track), and MSER (Maximally Stable Extremal Regions).

A number of feature points are detected and classified in the reference image (FIG. 18A) and the test image (FIG. 18B), respectively. Each circle in the image represents a feature point, and the size of the circle represents a scale of the identified point. Feature points are identified in scale-space utilizing the determinant of the Hessian (DoH). A mask determined from color segmentation may be used to filter out any SURF features detected inside the iris of the eye. The remaining features may be matched by means of a random sampling method, such as the random sample consensus method (RANSAC), to enable a large number of outliers. The RANSAC method computes a homography from a minimal subset of feature points, and randomly adds features to find the solution, which encompasses the largest number of feature points. The resulting homography may then be transformed into a camera position and orientation, as shown in FIG. 19, and compared to the known input transformation.

In another embodiment, the processing unit may analyze 2D images generated by the cameras based on parameters of the cameras and a spatial relationship between the cameras. The processing unit may then compare the 2D analyses or use a 3D registration to calculate the position of the microscope focal plane relative to a plane of interest with respect to the real object. For example, the processing unit may determine the distance between the microscope focal plane and the plane of interest. The processing unit may then use that distance to adjust the focus of the projected images generated by the insert so as to match the plane of interest with respect to the real object. This technique provides the benefit of relieving eye strain of the surgeon when viewing the projected images and the analog image of the object at the same time by focusing the projected images to the plane of interest that the user is visualizing.

Based on the registration and tracking discussed above, the processing unit may then control the display devices to adjust the overlaid images to track the changes or motions of the object viewed under the microscope, to display information relevant to the motions of the object, or perform other functions accordingly. For example, in cataract surgery, once a patient's eye has been registered, the processing unit may cause the insert to render tags and labels associated with individual layers or features of the eye, such as the sclera, limbus, pupil, or iris, or other very small layers of the eye. The processing unit may identify different anatomical layers or features of the eye and generate graphical elements in the overlaid images according to the identified anatomical layers or features.

FIG. 20 illustrates a sequence diagram for interactions between a tracker engine configured to track the motions of an eye and a torsion engine configured to generate graphical representations for the guidance or prompts. FIG. 21 illustrates an exemplary tracking system for tracking the eye of a patient and generating the graphical representations for the guidance or prompts.

FIG. 22 illustrates an exemplary process for carrying out the tracking by the tracker engine of FIG. 21. The main processing blocks are similar for both the reference and sense images, and are illustrated in FIG. 22. These are detailed as follows:

Image Preprocessing—pre-filtering the reference and sense images prior to edge detection.

Edge Detection—Canny edge detector.

Generalized Hough Transform—a modified Hough Transform is used the segment the limbic boundary.

Define and unwrap Annulus Region: The center and radius of the previously detected limbic boundary are used to define an annulus in the sceleral region. The annulus is then mapped from the Cartesian plane to the polar plane. The effect of this transformation is to map all pixels within the annulus to pixels in a rectangular region. i.e., f: (x, y) [symbol] (ρ, θ). The transformed annulus is sent to the Torsion Engine where the Occular Torsion angle in computed.

Finally, the tracker engine then passes the computed Occular Torsion angle along with the radius and center of the limbic boundary to the Render Engine.

FIG. 23 illustrates an exemplary process for reference image processing and generating Housdorff Distance look-up table. The main processing blocks are illustrated in FIG. 23 and are detailed as follows:

Image pre-processing—this processing step includes Histogram equalization.

Gabor filtering and Skeletonization, including extracting the vasculatures from the sclera using four orientations of a 2-D Gabor filter, skeletonizing the four feature images, combining (logical OR) the four skeletonized images, and skeletonizing the final image.

Define template ROI in reference annulus—a template ROI is extracted from the Gabor filtered and skeletonized reference annulus.

Compute table of all possible Hausdorff Distances—a look-up table is created at this step. It is predicated on the assumption that all pixels in the sense image ROI belong to a feature. This facilitates the computation of all possible Hausdorff Distances between the sense image ROI and the previously defined reference annulus template ROI.

Cache look-up table.

FIG. 24 illustrates an exemplary process for processing the sense image and computation of the minimum-Hausdorff Distance between the reference image template ROI and ROI in the sense image. The main processing blocks are illustrated in FIG. 24 and are detailed as follows:

Image pre-processing—this processing step includes histogram equalization.

Gabor filtering and Skeletonization, including extracting the vasculatures from the sclera using four orientations of a 2-D Gabor filter, skeletonizing the four feature images, combining (logical OR) the four skeletonized images, and skeletonizing the final image.

Define ROI in reference annulus—a ROI is extracted from the Gabor filtered and skeletonized sense image annulus,

Compute Hausdorff Distances—the minimum Hausdorff Distances between the sense image ROI and the previously defined reference annulus template ROI is computed.

Cache look-up table.

Compute the Occular Torsion angle.

FIGS. 25-32 depict a process for estimation of ocular torsion from sclera features. The ocular torsion estimation is based on the following assumptions:

The Ocular Torsion is bounded: |θ_{OT max}|≦20°

Between neighboring (successive) frames: θ_{OT}<2° typically |θ_{OT max}|∈[0.5, 2].

As shown in FIG. 25, torsion angle is deterministic. The computed angle is corrupted with additive noise (AWGN) and can be modeled as a sinusoidal (with AWGN).

During the estimation, at startup (i.e., the first frame), θ_{OT} is not known a-priori. It is assumed initially that |θ_{OT}|=20°. Worst case search scenario is that it may be needed to search the entire exemplar with high computational complexity. It is further assumed that the torsional rotation between subsequent frames is constrained |θ_{OT max}|∈[0.5, 2]. Only a small window is needed from the exemplar to compute the distance metric, and, hence, the computational complexity decreases.

During initialization of the estimation, the system captures exemplar frame from which to compute the reference feature vector. A reference image is captured with the subject (i.e., the patient) looking straight ahead. This is the exemplar image from which the feature template is generated.

According to an embodiment, the pupil radius of the patient under dilation is non-deterministic. The system may use a-priori knowledge of the pupil center and radius to determine an accurate estimate of the iris radius. The system may also preclude instabilities that may arise from extreme pupil dilation. Extreme pupil dilation precludes a safe guess as to the annular region which contains the outer iris boundary.

According to an embodiment, the system also computes iris radius. Iris boundary is deformable under incident forces. Absent any deforming incident forces, the radius is constant within bounds. The system first computes the pupil radius, which is non-deterministic under dilation, and precludes a “guess” for the annular ring which encloses the elliptical boundary of the iris. The system then computes iris radius.

During iris radius, the system may relax the stringent requirements that are necessary for Bio-metric applications. Iris is enclosed by the lumbus. The system may use an empirical determination of the max thickness of the lumbus, or the best fit circle for the iris. The inner radius, r₁, of the annulus is computed as follows: r₁=rᵢᵣᵢₛ+Δ, where Δ is the max possible thickness of the lumbus

FIG. 26 illustrates an exemplary process for iris boundary estimation, including frame capture, down sampling, histogram equalization, grey scaling, Gaussian kernel filtering, pupil center and radius calculation, iris center and radius calculation, and definition of annular region in sclera.

FIG. 27 illustrates an exemplary process for feature extraction from annular ring in exemplar image, including pre-definition of annular region in sclera, mapping annulus to rectangular region, sclera feature detection within (0−2π), and post processing. The sclera feature detection may be based on simple edge detection, such as Sobel filter, Canny filter, etc., Gabor wavelet, or wavelet using the lifting scheme,

FIG. 28 illustrates an exemplary feature extraction from segment of annular ring in test image, including segmentation from pre-defined annular region in sclera, mapping annular segment to rectangular region, sclera feature detection, and post processing. The pre-determined segment is taken from the pre-defined annular ring. The annular ring may be previously defined in the exemplar image. The segmentation is included by two angles θ₁, θ₂ ∈[0, 2π].

FIG. 29 illustrates an exemplary annular ring constructed in scleral region of an exemplar image. FIG. 30 illustrates an exemplary mapping of annulus in sclera to a rectangular region.

FIG. 31 illustrates an exemplary segmentation in annular region in a test image. FIG. 32 illustrates an exemplary template matching process.

According to an embodiment, the estimation of the ocular torsion may be based on the city-block distance metric (i.e., l₁ norm). In particular, for an n-dimensional vector space, the system may calculate the l₁ norm between p and q by:

D_{pq}(p−q)=∥(p−q)∥₁=Σᵢ₌₁ⁿ|(pᵢ−qᵢ)|;

Where p=(pᵢ, p₂, . . . pₙ) and q=(q₁, q₂, . . . qₙ).

The l₁ norm metric is computationally more efficient than the Euclidean distance and the cross-correlation techniques.

According to an embodiment, the segments of the annular ring will, at times, be occluded during surgery. The system may choose a segment that is not occluded, as segment occlusion will lead to template match failure.

The system may mitigate the occlusion by locating the occluded segment. The occulated segment may be non-deterministic if a-priori knowledge of the location of the instruments is unknow. The computational complexity may also be high.

The system may also define a number of segments using a-priori knowledge of instruments location (if known) so as to preclude a choice of an occluded segment. Alternatively, the system may choose a segment randomly and compute the distance metric. The system will know, if failure, without need to search the entire exemplar since the torsion angle between neighboring frames is constrained—between neighboring (successive) frames: θ_{OT}<2° typically |θ_{OT max}|∈[0.5, 2].

Using a-priori knowledge of the torsion frequency will allow the system to compute the next torsion angle gradient, and will allow some limited adaptation. It will also allow estimation of the torsion frequency, when the pupil is dilated and the patient is prone. This is the preamble to actual surgery. The system may use best fit sinusoidal to torsion angles.

One of the benefits of using 3D registration based on two cameras in the disclosed microscope insert is that the microscope insert allows the processing unit to acquire depth information along the z axis of any anatomical features, which is not available in existing systems. The processing unit may the use the depth information to provide 3D guidance and assist the surgeon to navigate during the surgical procedure.

This disclosure is not limited to the particular implementations listed above. Other display techniques, protocols, formats, and signals may also be used without deviating from the principle of this disclosure. Other embodiments of the invention will be apparent to those skilled in the art from consideration of the specification and practice of the invention disclosed herein. Although the microscope insert is described above in the context of a cataract surgery, one of ordinary skill in the art will appreciate that the microscope insert may be integrated in other surgical systems configured to carry out a wild variety of surgical procedures, such as spinal surgery, ear, nose, and throat (ENT) surgery, neurosurgery, plastic and reconstructive surgery, gynecology, oncology, etc. For these procedures, the insert may be used for registration, tracking, and image recognition and to generate customized stereoscopic overlaid information relevant to the procedure and a particular patient's anatomy that is not limited to what is disclosed herein. It is intended that the specification and examples be considered as exemplary only, with a true scope and spirit of the invention being indicated by the following claims.

What is claimed is:

1. A microscope insert, comprising:
a camera configured to receive a first portion of first light through a microscope from an object and generate a signal representing an image of the object;
a display device configured to generate a graphical representation of information relevant to the object and project second light representing the graphical representation;
a beam splitter configured to direct a second portion of the first light from the object and a first portion of the second light to a viewing device for simultaneously viewing the object and the information by a user; and
a processing unit configured to track motions of the object based on the image of the object and control the display device to adjust the graphical representation according to the motions of the object.

2. The microscope insert of claim 1, wherein the processing unit tracks the motions of the object based on fiducial markers placed on the object.

3. The microscope insert of claim 1, wherein the processing unit tracks the motions of the object based on anatomical features of the object.

4. The microscope insert of claim 1, wherein the processing unit is further configured to:
determine a focal plane of the microscope based on the image of the object; and
adjust a focus of the graphical representation according to the focal plane of the microscope.

5. The microscope insert of claim 1, wherein the beam splitter is configured to direct a second portion of the second light to the camera, and the camera is configured to capture an image of the graphical representation generated by the display device based on the second portion of the second light.

6. The microscope insert of claim 1, wherein the processing unit is configured to determine a movement of the object based on the image of the object.

7. The microscope insert of claim 6, wherein the processing unit is configured to determine three-dimensional position and orientation of the object.

8. The microscope insert of claim 6, wherein the processing unit is configured to determine coordinate data corresponding to at least one of movement, position, or orientation of the object.

9. The microscope insert of claim 8, wherein the processing unit is configured to adjust the graphical representation generated by the display device according to the coordinate data.

10. The microscope insert of claim 8, wherein the object includes an eye, and the coordinate data include at least one of a coordinate of a center of the eye, a radius of a limbus of the eye, or a rotational angle of the eye.

11. The microscope insert of claim 10, wherein the coordinate of the center of the eye and the radius of the limbus of the eye are represented in pixels.

12. The microscope insert of claim 1, wherein the processing unit is configured to enhance contrast of the image of the object.

13. The microscope insert of claim 12, wherein the processing unit is configured to perform segmentation of the image of the object.

14. The microscope insert of claim 13, wherein the processing unit is configured to generate a binary mask corresponding to the object based on the segmentation of the image of the object.

15. A method for tracking and registering an object in a microscope, comprising:
receiving first light from an object through a microscope;
generating, based on a first portion of the first light, a first signal representing an image of the object;
generating, according to the image of the object, a graphical representation of information relevant to the object;
projecting second light corresponding to the graphical representation of the information;
directing a second portion of the first light from the object and a first portion of the second light to a viewing device for simultaneously viewing the object and the information by a user;
tracking the object based on the image of the object; and
adjusting the graphical representation according to the tracking of the object.

16. The method of claim 15, further comprising:
generating, based on a second portion of the second light, a second signal representing an image of the graphical representation; and
adjusting a focus of the graphical representation based on the image of the object and the image of the graphical representation.

17. The method of claim 15, wherein the tracking of the object further comprises determining coordinate information of the object based on the image of the object.

18. The method of claim 17, wherein the object comprises an eye, and the coordinate information comprises at least one of a coordinate of a center of the eye, a radius of a limbus of the eye, or a rotational angel of the eye.

19. The method of claim 17, wherein the adjusting of the graphical representation further comprises applying a coordinate transformation to the graphical representation according to the coordinate information of the object.

20. The method of claim 17, further comprising determining the coordinate information based on one of anatomical features of the object or markers disposed on the object.
